(19) United States
(12) Patent Application Publication
(10) Pub. No.: US 2014/0315259 A1
(43) Pub. Date: Oct. 23, 2014

(54) CELLULOSE CO-FEED FOR DRY MILL CORN ETHANOL OPERATIONS

(71) Applicant: Edeniq, Inc., Visalia, CA (US)

(72) Inventors: Richard Root Woods, Three Rivers, CA (US); James Kacmar, Visalia, CA (US)

(21) Appl. No.: 14/211,450

(22) Filed: Mar. 14, 2014

Related U.S. Application Data
(60) Provisional application No. 61/799,081, filed on Mar. 15, 2013.

(51) Int. Cl. C12P 7/10; C12P 19/02; C12P 19/14

(52) U.S. Cl. CPC C12P 7/10; C12P 19/14; C12P 19/02
USPC 435/99; 435/165; 435/145; 435/160; 435/157; 435/167; 435/166; 435/140; 435/139; 435/136

(57) ABSTRACT

The present application provide methods for producing ethanol from a biomass. The methods combine sugars produced from a feedstock containing starch with sugars produced from a cellulosic biomass. The methods allow increased amounts of ethanol to be produced from a given solids concentration in the fermenters. The methods also encompass filtering the liquefied feedstock mash through a filter comprising biomass fibers. The biomass filter produces a post-filtered mash stream comprising a high concentration of sugars and a low concentration of non-fermentable solids. The methods provide numerous advantages described herein.

### CROSS-REFERENCE TO RELATED PATENT APPLICATIONS

The present application claims benefit of priority to U.S. Provisional Patent Application No. 61/799,081, filed Mar. 15, 2013, which is incorporated by reference herein in its entirety.

### BACKGROUND OF THE INVENTION

In a conventional ethanol facility, the fiber content of the corn kernel biomass is currently not hydrolyzed into fermentable sugars and passes through the fermentation and distillation stages as non-fermentable solids. Corn biomass typically consists of endosperm (high in starch), germ (high in oil and fiber), bran (high in fiber), and the corn tip (high in fiber). The non-fermentable solids create several problems that lower the efficiency and/or decrease the quality of downstream products. For example, the extra solids decrease the protein and fat content of the dried distillers grains (DDG) co-products.

The total fermentable and non-fermentable solids also provide an upper limit on the amount of feedstock that can be processed for fermentation. Corn ethanol plants typically operate with 30 to 35% weight by weight (w/w) solids in the fermenters, of which 65 to 75% is starch. However, increasing the dissolved solids concentrations above about 30-35% w/w results in decreased yeast efficiency due to osmotic stress induced by the increased concentration of very small suspended particles and dissolved compounds. The decreased yeast performance results in a tradeoff between throughput ethanol production in gallons and yield (process efficiencies) in gallons per bushel of COM.

Ethanol can also be produced from cellulosic biomass feedstocks. However, co-feeding a cellulosic biomass source, such as corn stover or corn cobs, directly into the front end of a conventional ethanol facility will increase the total solids that are loaded into the fermenters and affect yeast performance. Because 95-98% of the starch that is present in corn flour is consumed by the yeast and 90 to 93% is converted to ethanol, directly adding additional cellulose fiber solids will impact process efficiency and can decrease both the throughput and the yield of the facility. As a result most conventional approaches to combining the two feedstocks utilize parallel processing trains dedicated to a specific feedstock. The present application provides methods for increasing ethanol production and yield by providing a single fermentation stream having a high sugar concentration from a combination of cellulosic and non-cellulosic biomass, coupled with a low non-fermentable solids concentration.

### BRIEF SUMMARY OF THE INVENTION

Methods are provided for producing ethanol from both a feedstock comprising starch and/or fermentable sugars and a biomass fiber comprising sugars derived from cellulose. The methods allow for increased ethanol production from a given concentration of feedstock solids by “co-feeding” the cellulosic biomass into the feedstock stream, such that additional sugars produced from the cellulosic biomass are combined with sugars from the starch prior to fermentation. The methods also allow the amount of feedstock comprising starch to be reduced in the upstream feed stream, without a corresponding reduction in ethanol production, by replacing the sugars produced from starch with sugars produced from the cellulosic biomass. In some embodiments, the methods described herein produce a fermentation stream having a high sugar concentration and a low non-fermentable solids concentration.

Thus, in one aspect, a method is provided for processing a cellulosic biomass, the method comprising:
a) generating a liquefied mash from a feedstock comprising non-cellulosic biomass;
b) filtering the liquefied mash through the cellulosic biomass to generate a first liquids stream comprising dissolved sugars and a first solids stream comprising the cellulosic biomass and non-dissolved components from the liquefied mash;
c) treating the first solids stream under conditions sufficient to convert components of the biomass to cellulosic sugars, thereby producing a mixture comprising solids, liquids, and dissolved cellulosic sugars;
d) separating the mixture into a second liquids stream comprising dissolved sugars and a second solids stream;
e) contacting the second liquids stream with feedstock to form a slurry; and
f) processing the slurry to produce liquefied mash, thereby producing a mash comprising both cellulosic and non-cellulosic sugars.

In some embodiments the process is continuous and the initial feedstock added during process initiation is used to generate a first liquefied mash. The first liquefied mash is processed according to steps (b), (c), and (d) of the method to produce the second liquids stream comprising sugars derived from cellulosic biomass. The second liquids stream is mixed with feedstock to produce a second liquefied mash. The second liquefied mash comprises sugars derived from the initial feedstock, which typically comprises starch, and sugars derived from the cellulosic biomass. In some embodiments the second liquefied mass has the same components as the first liquefied mash. For example, in a continuous steady-state process, the liquefied mash typically comprises non-cellulosic sugars and cellulosic sugars.

In some embodiments, the method further comprises processing the first liquid stream under conditions suitable to produce a product from the sugars and a whole stillage stream. In one embodiment, the product is a concentrated sugar stream. In one embodiment, the product is a chemical. In some embodiments, the first liquids stream is fermented to produce the product. In some embodiments, the product is ethanol, succinic acid, butanol(s), methanol, propanol(s), isoprene(s), aromatics, farnesene, acetic acid, lactic acid(s), or levulinic acid(s). In some embodiments, the ethanol is removed using a per-vaporization membrane.

In some embodiments, the method further comprises processing the whole stillage stream to generate a third liquids stream and a third solids stream, wherein a portion of the third liquids stream and the first solids stream are mixed under conditions suitable to convert components of the biomass to sugars. In some embodiments, water is recovered from at least a portion of the third liquids stream and the water is mixed with the first solids stream under conditions suitable to convert components of the biomass to sugars.

In some embodiments, the method further comprises recovering an oil co-product from the mixture.

In some embodiments, the mixture comprising solids, liquids, and dissolved cellulosic sugars is treated with a high shear reactor. In some embodiments, the mixture is separated into the second liquids stream and the second solids stream using a centrifuge, a membrane, or a filter.

In some embodiments, the filtering step comprises filtering the mash through biomass comprising fiber. In one embodiment, the liquefied mash is filtered through a device employing the cellulosic biomass as a filter medium. In some embodiments, the method further comprises separating the liquefied mash into a filtrate comprising sugars and a retentate comprising solids and enzymes.

In some embodiments, the method further comprises washing the second solids stream with an aqueous solution and adding the post-wash aqueous solution to the slurry. In some embodiments, the method further comprises contacting at least a portion of the third liquids stream with the first solids stream prior to or during step (c) and/or step (d). In one embodiment, the method further comprises evaporating a portion of the third liquids stream to produce a water condensate that can be used in step (c) or (d) or in the step of washing the second solids stream with an aqueous solution.

In another aspect, a method is provided for producing ethanol from a cellulosic biomass in an ethanol facility, the method comprising:
a) separating the whole stillage into a first liquid stream and a first solids stream;
b) contacting the cellulosic biomass with at least a portion of the first liquid stream under conditions suitable to convert components of the biomass to sugars, thereby producing a mixture comprising solids, liquids and dissolved cellulosic sugars;
c) contacting the mixture with additional feedstock comprising non-cellulosic biomass to form a slurry; and
d) processing the slurry under conditions sufficient to produce ethanol, thereby co-producing ethanol from the cellulosic sugars and the non-cellulosic feedstock.

In some embodiments, the method further comprises (e) recovering water from at least a portion of the first liquid stream, and (f) contacting the cellulosic biomass with the recovered water under conditions suitable to convert components of the biomass to sugars, thereby producing a mixture comprising solids, liquids and dissolved cellulosic sugars.

In some embodiments, the method further comprises (g) separating the mixture into a second liquid stream comprising fermentable sugars and a second solids stream comprising non-converted biomass, and (h) contacting the second liquid stream with additional feedstock comprising non-cellulosic biomass to form the slurry. In some embodiments the concentration of sugars achievable in the slurry is greater than the concentration of sugars achievable in the mixture or the second liquid stream.

In some embodiments, the method further comprises washing the second solids stream with an aqueous solution and adding the post-wash aqueous solution to the slurry. In one embodiment, a portion of the second solids stream is contacted with the feedstock or non-cellulosic biomass under conditions suitable to convert components of the biomass to sugars, thereby producing sugars. In some embodiments, the concentration of sugars is greater than the concentration of sugars achievable

In some embodiments, the cellulosic biomass comprises corn stover, wheat straw, bagasse, wood or any other cellulosic fiber. In one embodiment, the cellulosic biomass is pretreated. In one embodiment, the cellulosic biomass is not pretreated.

In some embodiments, the feedstock comprises corn, wheat, milo, rice, barley, sugar cane, sugar beets, tubers, or Jerusalem artichokes. In some embodiments, the feedstock comprises starch and/or fermentable sugars.

In some embodiments, the biomass and/or mixture is treated with a high shear reactor. In some embodiments, the conditions suitable to convert include contacting the biomass with enzymes comprising cellulases such that the enzymes hydrolyze at least a portion of the biomass to sugars.

### DEFINITIONS

Unless defined otherwise, all technical and scientific terms used herein have the same meaning as commonly understood by one of ordinary skill in the art to which this disclosure pertains. Although essentially any methods and materials similar to those described herein can be used in the practice or testing of the present disclosure, only exemplary methods and materials are described. For purposes of the present disclosure, the following terms are defined below.

The terms “a,” “an,” and “the” include plural referents, unless the context clearly indicates otherwise.

The term “dissolved solids” refers to soluble compounds comprising sugars, soluble carbohydrates, polysaccharides, residual lignin, and other such substances. The term can include solids that are not retained by solid-liquid separation methods. Exemplary solid-liquid separation methods include, but are not limited to, filtration, membrane filtration, tangential flow filtration (TFF), centrifugation, sedimentation and flotation.

The term “conditions suitable to convert components of the biomass to sugars” refers to contacting the solids phase biomass with hydrolytic enzymes including, but not limited to, one or more cellulase(s), hemicellulose(s) and auxiliary enzyme(s) or protein(s), singly or in any combination, in order to produce fermentable sugars from polysaccharides in the biomass. The conditions can further include a pH that is optimal for the activity of saccharification enzymes, for example, a pH range of about 4.0 to 7.0. The conditions can further include a temperature that is optimal for the activity of saccharification enzymes, for example, a temperature range of about 15° C. to 100° C. based on the stability of the enzymes used. The term “conditions suitable to convert components of the biomass to sugars” also includes other non-enzymatic methods of hydrolysis by strong and weak acids, by high temperature hydrolysis, and by other reactive compounds, as well as by a combination of any one or more of these methods with the application of high shear forces.

The term “permeate” refers to the liquid or fluid that passes through a porous membrane or filter. If a filter is used, the term is synonymous with “filtrate”.

The term “retentate” refers to the material that does not pass through a porous membrane or filter, and is thereby retained by the membrane or filter.

The term “biomass” in its broadest sense refers to any material derived from a plant. The term “biomass fibers” or “cellulosic biomass” refers to any material comprising primarily lignocellulosic material. Lignocellulosic materials are composed of three main components: cellulose, hemicellulose, and lignin. Cellulose and hemicellulose contain carbohydrates including polysaccharides and oligosaccharides, and can be combined with additional components, such as protein and/or lipid. Examples of cellulosic biomass include but are not limited to agricultural products such as corn stover, corn cobs and other inedible waste parts of food plants; bagasse which is the fiber material of sugarcane after the free sugars have been removed; food waste; agricultural residues; grasses such as switchgrass; forestry biomass, such as wood, paper, board and waste wood products, and components of municipal waste materials. The term “lignocellulosic” refers to material comprising both lignin and cellulose, and may also contain hemicellulose.

The term “cellulosic,” in reference to a material or composition, refers to a material comprising cellulose, and may also contain hemicellulose. Specifically, cellulose is a polymer backbone comprised of beta 1-4 linked glucose residues and hemicellulose is a polymer backbone comprised of xylan (beta 1-4 linked D-xylose), mannan (beta 1-4 linked D mannose) and xyloglucan and hydrocarbons with branches composed of D-galactose, D-xylose, L-arabinose, and D-glucuronic acid. Composition is dependent on plant species and tissue, and therefore, varies with different biomass sources.

The term “feedstock comprising non-cellulosic biomass” or “non-cellulosic feedstock” refers to a biomass feedstock that comprises starch or other sources of non-cellulosic sugars, including but not limited to flour from grains such as corn, wheat, barley, and milo and to other feedstock such as sugarcane, sugar beets, sunflowers (e.g., tubers of the Jerusalem artichokes), and other biomass primarily used as a source of sugars and short chain sugar oligomers.

The term “saccharification,” also referred to as “hydrolysis,” refers to production of sugars and short chain sugar oligomers from biomass or biomass feedstock or feedstock comprising non-cellulosic biomass. Saccharification can be accomplished by saccharification or hydrolytic enzymes, cellulases, alpha amylases, gluco-amylases, beta gluco-amylases, and/or auxiliary proteins, including, but not limited to, peroxidases, laccases, expansins and swollenins “Hydrolysis” refers to breaking the glycosidic bonds in polysaccharides and the incorporation of a water to yield simple monomeric and/or oligomeric sugars. For example, hydrolysis of cellulose produces the six carbon (C6) sugar such as glucose and glucose oligomers, whereas hydrolysis of hemicellulose produces both the five carbon (C5) sugars such as xylose and arabinose and the six carbon (C6) sugars such as galactose and mannose and various oligomers. Generating short chain cellulosic sugars from polymer cellulosic fibers and biomass can be achieved by a variety of techniques, processes, and or methods. For example, cellulose can be hydrolyzed with water to generate cellulosic sugars. Hydrolysis can be assisted and or accelerated with the use of hydrolytic enzymes, chemicals, mechanical shear, thermal and pressure environments, and or any combination of these techniques. Examples of hydrolytic enzymes include β-glucosidase, xylanase, cellulases and hemicellulases. Cellulase is a generic term for a multi-enzyme mixture including exo-cellobiohydrolases, endoglucanases and β-glucosidases which work in combination to hydrolyze cellulose to cellobiose and glucose. Examples of chemicals include strong acids, weak acids, weak bases, strong bases, ammonia, or other chemicals. Mechanical shear includes high shear orifice, cavitation, colloidal milling, and auger milling. Examples of high shear devices include but are not limited to orifice reactors, rotating colloidal-type mills, Silverson mixers, cavitation milling devices, or steam assisted hydro jet type mills. High shear devices include any device with a stationary stator and a rotating rotor positioned to maintain a physical gap between the rotor and the stator during operation such that a high shear zone is generated within this gap or along this gap.

The term “sugars” shall include mono-saccharides and short chain sugars or oligosaccharides (mono-saccharides, disaccharides, and tri-saccharides) and medium chain oligosaccharides (DP-4 to DP-20), unless specifically defined as glucose, xylose, etc. which refer only to the mono-saccharide versions.

The term “fermentable sugar” refers to a sugar that can be converted to ethanol or other products such as but not limited to methanol, butanols, propanols, succinic acid, and isoprene, during fermentation, for example during fermentation by yeast. For example, glucose is a fermentable sugar derived from hydrolysis of cellulose, whereas xylose, arabinose, mannose and galactose are fermentable sugars derived from hydrolysis of hemicellulose.

The term “convertible sugar” refers to a sugar or sugar oligomer that can be converted to “ethanol” or other “product(s)”. The term “product” or “products” refers to any concentrated sugar product or compound, which can be generated through the conversion of sugars by any method, such as but not limited to ethanol, methanol, butanol(s), propanol(s), succinic acid(s), and isoprene(s) during fermentation, or that can be converted to synthetic gases comprising hydrogen and carbon monoxide, which can be converted to fuels such as but not limited to naphtha, kerosene, gasoline, and diesel replacements, or chemical products, such as but not limited to waxes, acetic acid, formaldehydes, polyethylene, xylenes, alcohols, oxygenates, synthetic LPG, olefins, ammonia, fertilizers, industrial chemicals, fine chemicals, and petroleum replacements chemicals, and to electric power and other energy media.

The term “fermentation” refers to the conversion of the sugars into ethanol or other product(s) by way of yeast, bacteria, or other biological microorganisms. Sugars can also be converted to ethanol or other product(s) by non-fermentation processes such as gasification, reformation or other chemical reactions. Sugars can also be converted to a sugars based product such as molasses or crystalline sugar, which can be final products or intermediate product for moving the mixed cellulosic and non-cellulosic sugars to another location for further processing. For terminology simplification the term “fermentation” shall be used to include all of these fermentation and non-fermentation processes which transform a raw sugars mixture or “convertible sugars” into a product.

The term “simultaneous saccharification and fermentation” (SSF) refers to providing saccharification enzymes during the fermentation process. This is in contrast to the term “separate hydrolysis and fermentation” (SHF) steps.

The term “pretreatment” refers to treating the biomass with physical, thermal, chemical or biological means, or any combination thereof, to render the biomass more susceptible to hydrolysis, saccharification, or conversion to sugars and short chain sugar oligomers, for example, by saccharification enzymes. Pretreatment can comprise treating the biomass at elevated pressures and/or elevated temperatures. Pretreatment can further comprise physically mixing and/or milling the biomass in order to reduce the size of the biomass particles and to produce a uniform particle size. Devices that are useful for physical pretreatment of biomass include, e.g., a hammermill, shear mill, cavitation mill or colloid or other high-shear mill. An exemplary colloid mill is the Cellunator™ (Edeniq, Inc., Visalia, Calif.). The use of a high-shear colloid mill to both reduce particle size and produce a uniform particle size to improve ethanol yields is described in, for example, WO2010/025171, which is incorporated by reference herein in its entirety.

The term “pretreated biomass” refers to biomass that has been subjected to pretreatment to render the biomass more susceptible to conversion.

The term “elevated pressure,” in the context of a pretreatment step, refers to a pressure above atmospheric pressure (e.g., 1 atm at sea level) based on the elevation. When used in thermal pretreatment, the term includes pressures sufficient to be equal to or greater than the pressure associated with the steam pressure at any given temperature of the process.

The term “elevated temperature,” in the context of a pretreatment step, refers to a temperature above ambient temperature, for example at least 100, 110, 120, 130, 140, 150, 160, 170, 180, 190, 200, 220, 240, or 300 degrees C. or greater. When used in thermal pretreatment, the term includes temperatures sufficient to substantially increase the pressure in a closed system. For example, the temperature in a closed system can be increased such that the pressure is in equilibrium with the temperature of water/steam within the system.

The term “inhibitor” refers to a compound that inhibits the saccharification and/or fermentation process. For example, both cellobiose and glucose inhibit the activity of cellulase enzymes. For example, xylo-oligomers, xylanase inhibitor proteins (XIP), and xylose inhibit the activity of hemicellulases. Other inhibitors include sugar degradation products that result from pretreatment of lignocellulose and/or cellulose. Examples of other inhibitors include 2-furoic acid, 5-hydroxy methyl furfural (HMF), furfural, 4-hydroxybenzoic acid (HBA), syringic acid, vanillin, syringaldehyde, p-coumaric acid, ferulic acid, organic acids such as acetic acid, and phenolic compounds from the breakdown of lignin. These inhibitors can also inhibit fermentation by inhibiting the activity or desired functionality of yeast or other biological microorganisms.

The term “non-cellulosic sugars” refers to sugars generated from feedstock comprising sucrose, dextrose, maltose, starch, inulin, and other non-fiber matter

The term “non-cellulosic ethanol” or “non-cellulosic products” refers to ethanol or products generated from the “non-cellulosic sugar” content or the sucrose or starch content of feedstock, comprising the starch portion of corn kernels, rye, wheat, milo, rice, etc. or the sucrose portion of sugar cane, sugar beets, etc. This definition is intended to provide illustrative examples and does not exclude the starch or sucrose portions of other plants.

The term “post fermentation stream” refers to a mixture of biomass solids and liquids (often referred to as “mash”) that was used to produce ethanol or another product by a fermentation process or other conversion process that transforms the sugars into a downstream product. For convenience, the term post fermentation stream is used to define the stream after the sugars have been used to produce a product, but is not limited to fermentation processes and includes other sugar conversion processes such as gasification, reformation, or non-fermentation chemical reactions. The solids can be either from cellulosic biomass or a non-cellulosic feedstock and comprise non-converted sugars, non-converted biomass, proteins, lignins, fats, oils, ash, chemical and other compounds. The term “post ethanol production stream” refers to a mixture of biomass solids and liquids that was processed to remove ethanol or other product.

The term “whole stillage” refers to the balance of the post fermentation stream after recovery of the product ethanol or other product compound(s), wherein the ethanol or other product has been at least partially removed, either by distillation or other means. Typically, the whole stillage is passed downstream to a separation process to generate a liquid stream and a solids stream.

The term “backset” refers to a liquid stream produced by a separation device that is recycled to an upstream point in the facility. In an ethanol facility the term “centrate” refers to a liquid stream from a centrifuge that is separated from the whole stillage stream, but herein centrate is not limited to centrifuge type separations, and the “wet grains” refers to the solids stream that is also separated. The centrate stream can be divided into the backset stream, which is returned to an earlier point in the process or used to dilute the biomass at any desired step, and a “thin stillage” stream, which is typically processed in an evaporator train or otherwise concentrated to recover water and can be further processed to yield products such as oil or “syrup.” For convenience the term “post fermentation stream”, “backset”, “whole stillage”, “centrate”, “thin stillage”, and “syrup” that are typical for a fermentation ethanol facility, are used to define similar streams in other facilities that are not fermentation or that produce product(s) other than ethanol.

### BRIEF DESCRIPTION OF THE DRAWINGS

FIG. 1 shows a schematic diagram of one embodiment of the methods, described more fully herein.

FIG. 2 shows a schematic diagram of one embodiment of the methods, described more fully herein.

FIG. 3 shows a schematic diagram of one embodiment of the methods, described more fully herein.

FIG. 4 shows a schematic diagram of one embodiment of the methods, described more fully herein.

### DETAILED DESCRIPTION OF THE INVENTION

### Introduction

The present application provides methods for producing ethanol and other products from a cellulosic biomass. The methods surprisingly allow an ethanol plant to produce the same amount of ethanol from less feedstock by adding sugars generated from the cellulosic biomass while keeping the total solids content to acceptable levels during fermentation. The methods can be used to produce fermentable sugars from the cellulosic biomass. In some embodiments, the fermentable sugars from the cellulosic biomass are combined with fermentable sugars from a feedstock comprising non-cellulosic biomass, such as starch. The combined sugars can be processed under conditions sufficient to produce ethanol or other products. The methods thus allow an ethanol plant to process less of the feedstock in order to produce the same or substantially similar amounts of fermentable sugars and/or products. The methods thus provide the advantage of keeping the total solids content that enters the fermenters below levels that adversely impact the function of microorganisms, such as yeast or bacteria, which convert sugars to ethanol or other products. For example, without being bound by theory, it is believed high amounts of dissolved and suspended solids (e.g., greater than about 30-35% weight by volume) increase the osmotic pressure on yeast, which decreases the ability of the yeast to convert sugars to ethanol. In some embodiments, the methods involve co-feeding or co-processing a cellulosic biomass, including but not limited to corn stover, with corn flour to increase the cellulosic content of the feedstock, and thereby, increase the amount of sugars available for downstream products. The methods will now be described.

### I. Methods

In one aspect, methods are provided for producing ethanol from a cellulosic biomass in an ethanol facility. In one embodiment, the method comprises generating whole stillage from a post fermentation stream, and separating the whole stillage into a liquid stream A and a solids stream A. As used herein, the term “liquid stream” refers to a stream that comprises more aqueous liquid or water than solids, for example at least 50%, 60%, 70%, 80%, 90% or more of an aqueous fluid. The liquid stream has a fluid characteristic. The term “solids stream” refers to a stream that comprises more solids than the liquid stream and substantially more suspended or non-dissolved solids then present in the liquid stream. Depending on the dissolved and suspended solids level of the feed stream the solids stream has substantially less fluid characteristics than the liquid stream. The whole stillage can be separated into a liquid stream A and a solids stream A using any suitable device or method known in the art. Non-limiting examples for performing the separation step include mechanical and centrifugal separation, such as by centrifuges, decanter centrifuges, screen centrifuges, presses, vibrating screens, filters, or extruders. If processed by a centrifuge, the liquid stream A is sometimes referred to as a “centrate” steam and the solids stream A is sometimes referred to as “wet grains.” In some embodiments, the cellulosic biomass is contacted with at least a portion of the liquid stream A (sometimes referred to in the art as “backset”) under conditions suitable to convert components of the biomass to sugars, thereby producing a mixture comprising solids, liquids and dissolved cellulosic sugars.

In some embodiments, the conditions suitable to convert components of the biomass to sugars include hydrolysis and/or saccharification of the cellulose in the biomass. In some embodiments, the conditions suitable to convert components of the biomass to sugars include contacting the biomass with enzymes comprising cellulases such that the enzymes hydrolyze at least a portion of the biomass to sugars. Suitable conditions for hydrolysis and/or saccharification of the cellulosic biomass are further described below. The sugars that are produced from the biomass can be used for any desired downstream process, such as fermentation to ethanol or conversion to other products.

Hydrolysis and/or saccharification and/or conversion of the cellulosic biomass can occur in any suitable device. In some embodiments, the device is a hydrolysis tank. In some embodiments, the device is a high shear reactor, as described herein. In one embodiment, the device is an auger or a battery of augers arranged in various series and or parallel flow paths. The devices can be batch and/or continuous or a hybrid of batch and continuous.

In some embodiments, the mixture comprising solids, liquids and dissolved cellulosic sugars is contacted with a feedstock comprising non-cellulosic biomass to form a slurry. Examples of non-cellulosic biomass include, but are not limited to, grains such as corn, wheat, milo, rice, and barley, sunflowers (e.g. high inulin tubers of Jerusalem artichokes) flotation, and sugarcane or sugar beets. The feedstock can also include starch and/or fermentable sugars and include inulin and fermentable fructose. The fermentable sugars can be produce by pretreating the biomass that is used for the feedstock. Pretreatment conditions are further described below. The slurry is then processed under conditions sufficient to produce ethanol. The conditions sufficient to produce ethanol can include contacting the slurry with enzymes under conditions sufficient to produce a liquefied mash comprising fermentable sugars, and contacting the mash with yeast under conditions sufficient to ferment the mash to produce ethanol or other products. Conditions sufficient to produce a liquefied mash are described below, and can include the same conditions suitable to convert components of the biomass to sugars. Conditions sufficient to ferment the mash to produce ethanol are described below. Thus, the ethanol is produced from sugars derived from both cellulosic biomass and from non-cellulosic feedstock, which is referred to herein as co-production of ethanol from cellulosic and non-cellulosic sugars or co-production of other products from cellulosic and non-cellulosic sugars.

In a second aspect, the methods are provided for producing ethanol from a cellulosic biomass, comprising generating whole stillage from a post fermentation stream, and separating the whole stillage into a liquid stream A and a solids stream A, as described above. In some embodiments, water is recover from at least a portion of the liquid stream A, and the recovered water is contacted with the cellulosic biomass under conditions suitable to convert components of the biomass to sugars, thereby producing a mixture comprising solids, liquids and dissolved cellulosic sugars. The resulting mixture can be contacted with a feedstock comprising non-cellulosic feedstock to form a slurry. The slurry is then processed under conditions sufficient to produce ethanol, thereby co-producing ethanol or other products from the cellulosic sugars and the non-cellulosic sugars in the feedstock.

In the above aspects, the amount of cellulosic biomass that is processed by the methods is about 1%, 3%, 4%, 6%, 8%, 10%, 12%, 14%, 20% or greater of total biomass and feedstock that is used in the slurry. For example, in some embodiments the ratio of cellulosic biomass to non-cellulosic feedstock is about 0.01, 0.03, 0.04, 0.06, 0.08, 0.10, 0.12, 0.14, 0.20 or greater.

In the above aspect the amount of cellulosic biomass that is processed can be chosen by the total solids in the mixture to enhance the conversion of biomass fibers to cellulosic sugars. Conversion of cellulosic biomass to cellulosic sugars can be inhibited by various process conditions; additionally, the high totals solids concentration of the pre-conversion mixtures can be difficult to pump or transport. In some conversion processes, such as but not limited to enzymatic hydrolysis, the degree of conversion and/or rate of conversion can be inhibited by the concentration of sugars in the conversion mixture. The amount of cellulosic biomass that is processed by the methods is chosen by the desired total solids in the mixture and the available amount of backset and/or recovered water such that the cellulosic conversion to sugars process is enhanced.

In the above aspects, the maximum ratio of cellulosic biomass to non-cellulosic feedstock is chosen by minimizing the compositional impact on downstream co-products such as animal feed products. The baseline processes, such as but not limited to a dry mill corn ethanol fermentation, have developed markets and market values for co-products such as distillers grains, wet distillers grains, modified distillers grains, dried distillers grains, and dried distillers grains with syrup (DDGS). The market values for these co-products have been established based on conventional process efficiencies and co-product compositions. For example, DDGS with 8% residual starch, 30% protein, 13% fat, and 30% corn kernel fiber may command a market value of 85% of the price of corn. Process improvements, such as but not limited to the Cellunator™ (Edeniq, Inc., Visalia, Calif.) for increased starch efficiencies or Pathway™ (Edeniq, Inc., Visalia, Calif.) for corn kernel fiber conversion, increase the utilization of available starch and reduce the concentration of low value components, such as starch and fiber in the DDGS. These types of technologies result in modifying the DDGS compositions with lower residual starch and fiber concentrations and higher concentration of high value components such as protein and fats, but the market price fails to adjust for these improvements. In some embodiments the amount of cellulosic biomass to non-cellulosic feedstock is chosen to reduce the high value component concentrations, such as protein and fat, in the DDGS co-product to concentrations established by market priced values consistent with baseline processes. This is achieved by replacing the consumed starch and corn kernel fiber with non-fermentable components of the cellulosic biomass and/or non-converted cellulosic biomass.

In some embodiments, the mixture comprising solids, liquids and dissolved cellulosic sugars can be further separated into a liquid stream B comprising fermentable sugars and a solids stream B comprising non-converted biomass. The mixture can be separated using any means known in the art. Methods for separating biomass mixtures are described in more detail below, and include mechanical means such as flotation, centrifuge, filter or press, or membrane, or combination of these means. In some embodiments, the liquid stream B comprising fermentable sugars is contacted with a feedstock comprising non-cellulosic biomass to form a slurry. Thus, the liquid stream B provides additional sugars that can be added to the feedstock slurry without adding additional solids, thereby allowing less feedstock to be used while at the same time providing optimal amounts of sugars for fermentation. In one embodiment, the liquid stream B (e.g., from the hydrolyzed biomass mixture) comprises a range of about 1-8% weight/volume (w/v) sugars and/or oligomers, for example at least about 1%, 2%, 3%, 4%, 5%, 6%, 7%, 8% or more w/v sugars. In some embodiments, the feedstock comprising non-cellulosic biomass comprises at least about 5%, 10%, 15%, 20%, 25%, 30%, 35%, 40% or more w/v sugars. In some embodiments, the total amount of sugar in the final slurry is at least about 10%, 15%, 20%, 25%, 30%, 35%, 40%, 50%, or more w/v sugars. The desired sugars concentration in the final slurry is based on the process optimization of downstream fermentation, conversion, concentration, and/or crystallization processes.

In some embodiments, the solids stream B comprising non-converted biomass is washed with an aqueous solution and the post-wash aqueous solution is added to the liquid stream B and/or the slurry. This wash step recovers additional sugars from the solids stream that can be fermented or converted to ethanol or other products. The desired sugars concentration in the final slurry is based on the process optimization of downstream fermentation, conversion, concentration, and/or crystallization processes.

In some embodiments, the solids stream B, or a portion thereof, is contacted with the cellulosic biomass and/or the feedstock under condition suitable to convert components of the biomass to sugars. This allows for recycling of active saccharification enzymes that are associated with the non-converted biomass solids in the solids stream B. In some embodiments, the solids stream B, or a portion thereof, is dried to produce a product such as dried distillers grains.

The liquid stream A can also be separated into a second portion (referred to as “thin stillage”) and optionally evaporated to produce evaporated thin stillage. The evaporated thin stillage can be dried to produce a product or evaporated to produce a “syrup.” Frequently, the wet grains and syrup are dried together or separately to make various products such as “dried distillers grains” (“DDG”), or “dried distillers grains with syrup” or “dried distillers grains with solubles” (“DDGS”). In some embodiments, the recovered water from the evaporators is added to the biomass under conditions suitable to convert components of the cellulosic biomass to sugars. The water from the evaporators can also be recovered and added to the feedstock slurry comprising non-cellulosic biomass. In some embodiments, the thin stillage or evaporated thin stillage can be processed to recover oil.

In some embodiments, the thin stillage can be concentrated with various technologies, such as but not limited to filtration, micro-filtration, and reverse osmosis, to recover water from the thin stillage. This recovered water is contacted with the cellulosic biomass under conditions suitable to convert components of the biomass to sugars, thereby producing a mixture comprising solids, liquids and dissolved cellulosic sugars.

In some embodiments, the cellulosic biomass comprises corn stover, wheat straw, bagasse, wood, stalks, or any other cellulosic fiber. In some embodiments, the cellulosic biomass is pretreated to make components of the biomass, such as cellulose and hemicellulose, more susceptible to hydrolysis, saccharification, or conversion. Methods for pretreating cellulosic biomass are described in more detail below. In some embodiments, the cellulosic biomass is not pretreated.

In some embodiments, the cellulosic biomass and/or the mixture comprising solids, liquids, and dissolved cellulosic sugars is treated with a high shear reactor in order to mix the biomass solids with saccharification enzymes. The high shear mixing environment provides more efficient conversion of cellulosic biomass to sugars and/or increase the rate of conversion of cellulosic biomass to sugars. In some embodiments, the high shear reactor is an auger, an orifice reactor, a rotating colloidal-type mill, a Silverson mixer, cavitation milling device, or a steam assisted hydro jet type mill. In some embodiments the colloidal type mill comprises a rotor (i.e., rotating component) and a stator (i.e., stationary component) with a physical gap between the rotor and the stator providing a high shear zone. The surface of the rotor or the stator can be smooth or arranged with flow grooves of various widths, orientations, and depths to promote the material to enter and exit the shear zone. In one embodiment, the high shear reactor is an auger or plurality of augers in series and parallel arrangements. In one embodiment the high shear reactor is a plurality of devices, operating in series and selected from an orifice reactor, a rotating colloidal mill, a Silverson mixer, cavitation milling device, and/or steam assisted hydro jet type mill. If desired, the particle size of the biomass and or biomass mixture can be reduced to a relatively uniform particle size that increases the amount of sugars and/or sugar oligomers that are produced without producing extremely fine, non-reactive particles that are so small they create problems with pumping the hydrolyzed mixture, separations of liquid streams from solids streams, or increase the osmotic pressure on yeast in downstream processes, such as fermentation or conversion. Methods for producing a relatively uniform biomass particle size are described in WO2010/025171, and U.S. Pat. No. 8,563,282, which are incorporated by reference herein in their entirety. The cellulosic biomass and or mixture can be treated with the high shear reactor prior to separation step B described above that separates the mixture into liquid B and solids stream B.

In a third aspect, a method is provided for processing a cellulosic biomass. In this aspect, the method uses the cellulosic biomass itself as a medium for filtering the post liquefied mash. This method provides the surprising result of providing a post-mash stream with a high concentration of fermentable sugars that also has minimal non-fermentable solids (e.g. fiber), protein and oil content (i.e., a clarified liquid stream). The mash stream with a high concentration of fermentable sugars and low concentrations of non-fermentable solids is passed to the fermenters. In some embodiments, the method produces a filtered mash stream comprising greater than 70%, 80%, 90%, 95%, 96%, 97%, 98% and 99% of the fermentable sugars from the mash on a dry basis. Any non-recovered sugars are recycled back to the slurry process. The method can thus provide an increase in ethanol production at constant fermentation solid levels. In some embodiments, the method increases ethanol production by at least 1%, 3%, 5%, 10%, 15%, 20% or more compared to operations in which the mash is not treated according to the method.

Thus, in one embodiment, the method comprises generating a liquefied mash from a feedstock comprising non-cellulosic biomass, and filtering the liquefied mash through the cellulosic biomass to generate a liquids stream C comprising dissolved sugars and a solids stream C comprising the cellulosic biomass and non-dissolved components of the liquefied mash. The solids stream C is treated under conditions sufficient to convert components of the cellulosic biomass to cellulosic sugars, thereby producing a mixture comprising solids, liquids, and dissolved cellulosic sugars. The mixture is separated into a liquids stream B comprising dissolved sugars and a solids stream B. The liquids stream B is contacted with the feedstock to form a slurry, and the slurry is processed to produce the liquefied mash, thereby producing a mash comprising both cellulosic and non-cellulosic sugars.

In some embodiments of the above method, the liquefied mash is generated from a traditional corn kernel feedstock comprising starch and/or fermentable sugars. In some embodiments, the feedstock is from a dry mill corn ethanol facility. The method adds sugars derived from the cellulosic biomass to the feedstock slurry, which is then treated to produce a mash. Alternatively, the sugars derived from the cellulosic biomass are added to the mash tank in a continuous or batch process. The mash is then liquefied to generate a liquefied corn mash is then processed and filtered as described above, and the liquid stream C is fermented to produce ethanol from both the non-cellulosic and cellulosic sugars. This approach requires achieving a higher slurry solids level then in a baseline non-cellulosic feedstock processing facility, such that the concentrated slurry solids plus the relatively dilute sugars derived from the cellulosic biomass stream when mixed achieves the desired post liquefied sugars concentrations.

In some embodiments, the filtering step comprises filtering the mash through cellulosic biomass comprising fiber. The filtering step can be accomplished with a device comprising dry fiber or corn stover that functions as a filter medium and/or binder agent to separate the large, non-hydrolyzed corn flour particles, proteins, germ, oil and a majority of the non-soluble components of the feedstock from the sugar-rich filtrate stream. The filtering device can be a filter press, extruding press, vibrating filter press, extruder type press, Vincent-type press, belt filter press, vacuum filter press, cylinder press, sand-type filter, or any other appropriate device known in the art. In some embodiments, the filter produces a relatively clear liquid stream C comprising high amounts of dissolved sugars that can be directly fermented. In some embodiments, the liquid stream C comprises at least 10%, 20%, 30%, 40%, and 50%, sugars or sugar oligomers concentration based on the solids level of the liquefied mash. In some embodiments the biomass fibers are directly mixed with the post liquefied slurry stream, such that the biomass fibers act as a binding agent for the smaller suspended particles in the post liquefied mash, and the mixture is passed to a solid liquid separation C device such as but not limited to a dewatering device or extruder type press in which a large fraction of the solids are retained and the bulk of the liquid and dissolved solids are separated as a liquid stream C with minimum suspended solids. In these cases 50%, 60%, 70%, 80%, 90%, 95%, or greater of the suspended solids in the liquefied mash are retained with the fibers and the suspended solid fraction of the bulk liquid stream is greatly reduced.

The liquefied corn mash in a conventional dry mill ethanol facility typically comprises 30 to 35% w/w solids, of which about 85 to 98% is hydrolyzed corn flour. The hydrolyzed corn flour consists of about 3.5 to 4.5% oils, 8 to 10% proteins, 10 to 12% fibers, and 2 to 6% ash, and about 65 to 76% starch. About 20 to 40% of the starch is converted into short chain sugars or oligosaccharides (mono-saccharides, disaccharides, and tri-saccharides) and about 60-80% remains as medium chain oligosaccharides and very little as long chain poly-sugars. As the corn flour becomes hydrated and heated it becomes gelatinized resulting in a very thick, high viscosity material having a relatively thick consistency that readily clogs filters. As the starch becomes hydrolyzed into shorter chain poly-saccharides, the consistency thins out, but based on the degree of hydrolysis and the presents of soluble proteins, some long chain poly-sugars remain and the bulk material can be thick and sticky. The cellulosic biomass filter described herein has the advantage of removing the gelatinized material and other non-fermentable solids from the post-liquefied mash liquid stream and has the bulk thickness and open tortuous flow paths to capture the gelatinized material, fats, proteins, and fibers and allow the dissolved compounds including short and medium chain oligosaccharides to pass through into the liquid stream C. In a conventional dry mill ethanol facility, the solids in the liquefied mash are added to the fermenters. However, the addition of solids during fermentation creates disadvantages, as described herein. Thus, the methods described herein provide a liquefied mash or hydrolyzed biomass having low amounts of suspended, non-fermentable solids that can be passed into the fermentation process.

Filtering the post liquefied mixture of biomass to reduce the solids concentration provides the following surprising advantages of the methods. First, the liquid stream C added to the fermenters can contain a high level of dissolved sugars to total solids ratio, for example, at least about 0.80, 0.85, 0.90, 0.95 or greater dissolved sugars to total solids ratio can be achieved, which results in increased ethanol production through improved efficiencies, shorter cycle times, and lower non-fermentable solids in the mash. Thus, more ethanol can be produced from the same amount of non-cellulosic feedstock or higher throughputs can be achieved with fixed fermentation volumes. Second, the post-mash solids stream (retentate) comprising the non-hydrolyzed components of the corn flour, specifically the proteins, fats, and corn kernel fibers and the cellulosic material from the biomass fiber filter (i.e. solids stream C) can be subjected to a saccharification process or conversion process, which can comprise a high shear saccharification reaction zone that minimizes cycle time and maximizes cellulose conversion in a dilute or high solids stream. Third, the size of the milling and saccharification hardware can be reduced because the solids stream comprises a lower volume having a higher solids concentration. In another embodiment of the current method, the backset water and/or recovered water from the downstream of the fermentation processes is used to dilute and/or wash the post filtered solids stream for recovery of residual mash sugars and oligomers. Similarly, the backset water and/or recovered water from downstream of the fermentation processes are used to manage the saccharification processes, and to wash the post saccharification stream of released cellulosic sugars.

Fourth, the method supports upstream extraction and/or recovery of co-products such as corn oil and animal feed products. The solids stream or retentate from the filter step after being subjected to the saccharification becomes liquefied due to the hydrolysis of the cellulosic material. In some embodiments, in which the oil present in the corn germ is fully or partially extracted and/or released by enzymatic and/or mechanical and/or thermal disruption of the germ cell structures and in which the stream has decreased viscosity due to the saccharification of the cellulose and fiber components, the oil and/or oily emulsion can be recovery from the post-saccharified solids stream by various technologies. Upstream oil extraction provides the advantages of increased recovery and lower free fatty acid (FFA) compositions. For example, it is known that yeast can convert the triglycerides in corn oil to FFA during fermentation. Thus, the oil recovered pre-fermentation can have less FFA concentrations. In some embodiments the FFA concentration of the product oil is less than 6%, less than 5%, less than 3% or less than 2% or even as low as less than 1%. Oil extracted downstream from the concentrated thin stillage or syrup can have much greater FFA concentrations of greater than 8%, or 10% or even as high as 15% depending on the upstream process characteristics. Further, the present methods provide an increase in the amount oil recovered. For example, the oil in the feedstock is typically about 2 lb. oil/bushel of grain. In a conventional dry mill ethanol facility with corn oil recovery, the oil is recovered from the thin stillage after the decanter centrifuge which separates the whole stillage into the centrate stream and wet grains. About half of the oil remains bound in the germ cell structures and passes out of the system with the wet grains and about half (e.g., about 1 lb. oil/bushel of grain) passes out with the thin stillage after dividing the centrate into backset and thin stillage. Because of the integration of corn oil recovery with the thin stillage only half of the oil is recoverable. Therefore, in the present methods all of the oil remains with the post filter retentate which by-passes the fermentation or conversion process, and can be extracted—resulting in a commensurate, dramatic higher yield potential and or greater quality with lower FFA concentrations. The various saccharification processes and specifically the enzymatic hydrolysis of cellulose and hemicellulose disrupt the germ cell structure, lysing the cells, and release a large fraction of the oil. Conventional oil extraction and recovery technologies can achieve higher recovery rates because more of the oil has been released into the bulk stream.

Fifth, the method supports second stage fermentation of xylose (C5 sugars) downstream of the distillation of ethanol from glucose (C6 sugars) and co-fermentation or co-conversion of xylose and glucose fermenter or reactor. The low suspended solids of the fermentation feed stream supports both batch and continuous fermentation processes. When C6 fermentation is combined with in process ethanol recovery or extraction the continuous or staged sequential C5 conversion is further enhanced with or without the addition of additional C5 specific biological organisms. Sixth, because the clear fermentation liquid (i.e., liquid stream C) has minimal non-fermentable and/or suspended solids, the methods support the use of flash evaporation, pervaporation, and vapor compression distillation to minimize the amount of energy used to generate and/or purify ethanol. For example, ethanol can be recovered during fermentation using pervaporation technology and minimizing or eliminating fermentation cooling, as described herein. Seventh, the method supports yeast recycling with a post fermentation separation or decanter centrifuge step prior to higher temperature distillation steps to support active yeast recycling or yeast extract processing after distillation to support nutrient recovery and recycling to propagation or fermentation. Eighth, the method supports the rapid fermentation cycle times used in sugar cane ethanol facilities. With the ability to remove the non-fermentable solids and non-hydrolyzed long chain starch components from the fermentation feed stream and with the ability to manage yeast recycling, the typical 45 to 60 to 70 hour fermentation cycle of the corn ethanol plant can be reduced to under 40 hr, or under 35 hours, or under 30 hours. Typically, the first 24 to 30 hours of fermentation in a baseline corn ethanol plant the composition of sugar oligomers indicate that starch hydrolysis has not reached 98% completion and the rate of ethanol generation is very rapid limited by the yeast processing kinetics. The rate of ethanol generation between cycle times 30 hours and 60 hours is substantially lower due to sugars availability and ethanol inhibition. In this embodiment the long chain starch oligomers which are not hydrolyzed can be captured with the fibers and passed to the second stage fiber treatment steps for hydrolysis. The sugar stream feed to the fermentation has a higher composition of hydrolyzed sugars or short chain sugars and the ability to recycle active yeast provides the capability to increase ethanol generation kinetics and shorten fermentation cycle times. Ninth, the method can be modified to generate a low sugar concentration stream from the cellulosic biomass that can be directly measured for government credits such as Renewable Identification Numbers or RINs validation upstream of starch addition. The sugar concentration can be increased by adding starch from corn flour to produce a high sugar concentration for fermentation feed stream having minimal non-fermentable solids.

In some embodiments, the liquefied mash is separated into a filtrate comprising sugars and a retentate comprising solids and enzymes. For example, the mash can be filtered through the cellulosic fiber as described above to produce a high solids, high fiber retentate that is dosed with cellulase and xylanase enzymes, and treated with the high shear reactor to increase the saccharification rates of the cellulosic biomass.

In some embodiments, the method further comprises processing the liquids stream C under conditions suitable to produce a product from the sugars, and a whole stillage stream. In some embodiments, the liquids stream C is fermented to produce the product. In some embodiments, the product is ethanol, succinic acid, or butanol. In some embodiments, the ethanol is removed from the fermenters using a pervaporation membrane in the recycle loop of the fermenter because of the low suspended solids concentration in the fermentation mash. Evaporation of the ethanol under low pressure conditions on the gas side of a hydrophobic membrane extracts thermal energy from fermentation mash stream which in turn can reduce or eliminate the cooling load.

The whole stillage stream can be further processed to produce a liquids stream A (e.g., thin stillage) and a third solids stream A (e.g., “wet grains”). The solids stream A can be dried to produce a product such as distiller dried grains with solubles (DDGS). The liquids stream A, or a portion thereof, can be combined with the solids stream C and mixed under conditions suitable to convert components of the biomass in the solids stream to sugars. In some embodiments, the liquids stream A, or a portion thereof, is combined with the solids stream C prior to or during the step in which the biomass is converted to sugars. In some embodiments, the liquids stream A, or a portion thereof, is combined with the solids stream C prior to or during the step in which the hydrolyzed mixture is separated into the liquids stream B comprising dissolved sugars and the solids stream B.

In some embodiments, water is recovered from at least a portion of the liquids stream A, and the water is mixed with the solids stream C under conditions suitable to convert components of the biomass to sugars. For example, the thin stillage stream can be evaporated to produce evaporated thin stillage, and the water from the evaporators (also referred to as “cook water”) can be recovered and used to dilute the cellulosic biomass mixture during the hydrolysis treatment step. Thus, in one embodiment, at least a portion of the liquids stream A is evaporated to produce a water condensate that can be used to dilute to the biomass mixture either before or after hydrolysis or downstream separation steps. The water condensate can also be used to wash the solids stream B to recover additional sugars, and the post-wash solution added to the feedstock slurry.

In some embodiments, the mixture comprising solids, liquids, and dissolved cellulosic sugars is treated with a high shear reactor, as described above.

In some embodiments, the liquid stream C is processed to produce a concentrated sugar stream and a whole stillage stream. In one embodiment, the liquid stream C is processed to produce a chemical or other product and a whole stillage stream. For example the chemicals can be but are not limited to ethanol, methanol, butanol(s), propanol(s), succinic acid(s), and isoprene(s) during fermentation, or that can be converted to synthesis gases comprising hydrogen and carbon monoxide, which can be converted to fuels such as but not limited to naphtha, kerosene, gasoline, and diesel replacements, or chemical products, such as but not limited to waxes, acetic acid, formaldehydes, polyethylene, xylenes, alcohols, oxygenates, synthetic LPG, olefins, ammonia, fertilizers, industrial chemicals, fine chemicals, and petroleum replacements chemicals, and to electric power and other energy media.

In some embodiments, the method comprises recovering an oil co-product from the hydrolyzed mixture. For example, the oil co-product can be corn oil. The corn oil can be recovered from the mixture using mechanical, thermal, and/or chemical recovery technologies. One exemplary method for recovering oil is described in U.S. Pat. No. 8,236,977, which is incorporated by reference herein in its entirety. The mixture can be diluted with cook water to lower the solids content as necessary to extract the oil. The mixture can be treated with enzymes to disrupt the germ cell structures.

### A. Pretreatment

Prior to the processing steps described herein, the cellulosic biomass can be pretreated to render the lignocellulose and cellulose more susceptible to hydrolysis. Pretreatment includes treating the cellulosic biomass with physical, thermal, chemical or biological means, or any combination thereof, to render the cellulosic biomass more susceptible to hydrolysis, for example, by saccharification enzymes, or to render the biomass more susceptible to conversion into sugars and/or sugar oligomers. Examples of chemical pretreatment are known in the art, and include acid pretreatment, alkali pretreatment, ammonium pretreatment, supercritical extraction, etc. In some embodiments the pretreated cellulosic biomass can be washed or washed and pressed to extract organic acids and or inhibitors that can be generated by the pretreatment. If acid pretreatment is used the pH range of the slurry can be decreased to below 4, or below 3 or below 2 pH with the addition of various strong acids such as H2SO4, HCl, H3PO4, SO2, etc.

One example of physical pretreatment includes elevated temperature and elevated pressure without the addition of strong acids. Thus, in some embodiments, pretreatment comprises subjecting the biomass fibers to elevated temperatures and elevated pressure in order to render the lignocellulose and cellulose accessible to enzymatic hydrolysis. In some embodiments, the temperature and pressure are increased to amounts and for a time sufficient to render the cellulose susceptible to hydrolysis. In some embodiments, the pretreatment conditions can comprise a temperature in the range of about 150° C. to about 300° C., or about 150° C. to about 210° C., or about 165° C. to 195° C. The pretreatment temperature can be varied based on the duration of the pretreatment step. For example, for a pretreatment duration of about 20, 30, 40, 50, and 60 minutes, the temperature is about 160° C. to 180° C.; for a duration of 10, 20, and 30 minutes, the temperature is about 180° C. to 190° C.; for a duration of 5 to 10 minutes, the temperature is about 210 degrees C. After temperature exposure the pretreated material can be flashed or rapidly depressurized to disrupt the fiber structure. Another example includes exposure of the cellulosic biomass to steam an elevated temperatures and pressures with rapid pressurization and depressurization cycles and controlled temperature heating and cooling rates. Another example of physical/chemical pretreatment includes mild to medium pyrolysis pretreatment, in which the cellulosic biomass is conditioned over relatively complex temperature and humidity profile cycles that can include purge gas environments of reducing and/or oxidizing conditions.

The pretreatment conditions can also comprise increased pressure. For example, in some embodiments, the pressure can be at least 100 psig or greater, such as 110, 120, 130, 140, 150, 200, 250, 300 psig or greater. In some embodiments, the biomass fibers are pretreated in a closed system, and the temperature is increased in an amount sufficient to provide the desired pressure. In one embodiment, the temperature is increased in the closed system until the pressure is increased to about 125 to about 145 psig or increased to about 145 to 165 psig. Persons of skill in the art will understand that the temperature increase necessary to increase the pressure to the desired level will depend on various factors, such as the size of the closed system. In some embodiments, pretreatment comprises any other method known in the art that renders lignocellulose and cellulose more susceptible to hydrolysis, for example, acid treatment, alkali treatment, and steam treatment, or combinations thereof.

In some embodiments, the pretreatment step does not result in the production of a substantial amount of sugars. For example, in some embodiments, pretreatment results in the production of less than about 10%, 5%, 1%, 0.1%, 0.01%, or 0.001% by weight glucose, less than about 10%, 5%, 1%, 0.1%, 0.01%, or 0.001% by weight xylose, and/or less than about 10%, 5%, 1%, 0.1%, 0.01%, or 0.001% by weight total sugars in general. In some embodiments, the amount of sugars in the process stream entering the pretreatment stage is substantially the same as the amount of sugars in the process stream exiting the pretreatment stage. For example, in some embodiments, the difference between the amount of sugars in the process stream entering the pretreatment stage and the amount of sugars exiting the pretreatment stage is less than about 10%, 5%, 1%, 0.1%, 0.01%, or 0.001% by weight.

In some embodiments, pretreatment can further comprise physically mixing and/or milling the biomass fiber and/or the biomass feedstock in order to reduce the size of the biomass particles. The yield of biofuel (e.g., ethanol) can be improved by using biomass particles having relatively small sizes. Devices that are useful for physical pretreatment of biomass include, e.g., a hammermill, shear mill, cavitation mill, orifice reactors, colloid mills or other high shear mill. Thus, in some embodiments, the pretreatment step comprises physically treating biomass with a colloid mill. In some embodiments, the biomass is physically pretreated to produce particles having a relatively uniform particle size of less than about 1600 microns. For example, at least about 50%, 60%, 70%, 80%, 85%, 90%, or 95% of the pretreated biomass particles can have a particle size from about 100 microns to about 800 microns. In some embodiments, at least about 50%, 60%, 70%, 80%, 85%, 90%, or 95% of the pretreated biomass particles have a particle size from about 100 microns to about 500 microns. In some embodiments, the biomass is physically pretreated to produce particles having a relatively uniform particle size using a colloid mill. The use of a colloid mill to produce biomass particles having a relatively uniform particle size, e.g., from about 100 microns to about 800 microns, can result in increased yield of sugars, as described in U.S. Patent Application Publication 2010/0055741 (Galvez et al.) (now U.S. Pat. No. 8,563,282), which is incorporated by reference herein in its entirety.

In some embodiments, the pretreatment step does not involve the use of acids which can degrade sugars into inhibitors of fermentation.

In some embodiments, the pH of the pretreated biomass is adjusted to a pH of between about 3.0 and about 7 or between 4 and 6.5 or between 4.5 and 6.0. In some embodiments, the pH of the biomass is adjusted during or after the pretreatment step to be within the optimal range for activity of saccharification enzymes, e.g., within the range of about 4.0 to 7.0, about 4.0 to 6.5, about 4.0 to 6.0, about 4.5 to 6.0, about 4.0 to 5.5, about 4.5 to 5.5, or about 5.0 to 5.5. In some embodiments, the pH of the biomass is adjusted using Ca(OH)₂ or Mg(OH)₂, NH₄OH, NH₃, or a combination of Ca(OH)₂ or Mg(OH)₂ and NH₄OH or NH₃ or NaOH. In some embodiments the pH of the biomass can be initially raised to above a pH of 7, 8, 9, or greater to affect the lignin structures surrounding during cellulose and hemicellulose.

After pretreatment, the pretreated biomass is processed to produce sugars using the methods described herein. Non-limiting embodiments will now be described.

B. Exemplary Methods for Generating Sugar from Biomass

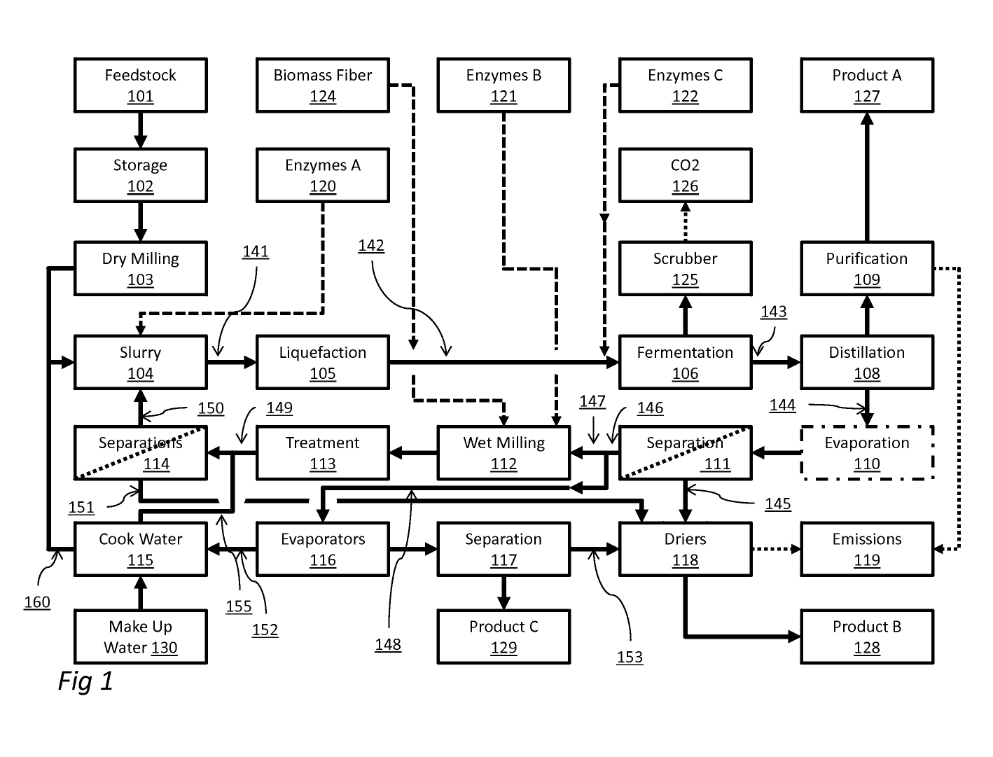

Referring now to FIG. 1, one embodiment will be described. A non-cellulosic feedstock 101 (which can be optionally stored in a storage unit 102) is milled 103 to a desired particle size and combined with an aqueous liquid 160 to form slurry 104. The slurry 104 can comprise, for example, about 30-35% w/w solids or greater than 35% solids. In some embodiments the slurry can be wet milled or treated with a high shear rotor stator device to maximize the liberation of starch and starch oligomers. In some embodiments, the feedstock 101 can comprise grains, including but not limited to corn, wheat, milo, rice, or barley. In some embodiments, the feedstock comprises sugar cane or sugar beets. In some embodiments, the feedstock comprises starch, fermentable sugars, fiber, and/or oil. The slurry stream 141 is treated 105 to convert the feedstock to a liquefied mash 142 comprising fermentable sugars including sugar oligomers. The treatment conditions include heating the slurry and adding enzymes 120 to the slurry that aid in converting starch to fermentable sugars and short and medium chain sugar oligomers. Suitable enzymes 120 include amylases such as alpha-amylases, fungal amylases, and others which hydrolyze the linkage bonds between glucose sugars along the starch polymers.

The liquefied mash 142 is then fermented 106 to produce ethanol using conditions well known in the art. The fermentation conditions typically include contacting the mash with yeast and enzymes 122 such as gluco-amylase. In some embodiments, cellulase enzymes are also added to the fermentation step to convert the cellulosic fiber in the feedstock to sugars. The ethanol is removed from the post-fermentation mash 143 by distillation 108, followed by purification 109 to result in Product A 127. If other products, e.g., non-ethanol products, are the target product of a conversion process 106 (labeled fermentation) then different extraction and/or recovery technologies might be used in place of distillation, and therefore, the specific product and/or method of product recovery and purification is not intended to limit the scope of the claims. The gaseous byproducts of fermentation can be sent to a scrubber 125 and carbon dioxide 126 recovered or vented. The remaining post-fermentation mash is referred to as whole stillage. The whole stillage stream 144 can be optionally evaporated 110 to remove water and concentrate the stillage or can be passed directly to the first separation process 111. The whole stillage stream 144 is then separated into a liquid stream A 146 and a solids stream A 145 by the first separation step 111 also known as decanter separation. The solid stream A 145 can be dried 118 to produce a product (Product B 128) such as DDGS or processed in other methods to achieve a co-product B 128. The separation step A 111 can be performed by any method known in the art, for example by mechanical devices such as a centrifuge, a decanter centrifuge, a press, or a filter, and the specific method of separation is not intended to limit the scope of the claims. If a centrifuge is used, the liquid stream A 146 is referred to as a centrate, but this term should not be limited to any specific mechanical device and is also defined as the liquid stream A. At least a portion of the liquid stream A 146 or centrate is combined with a cellulosic biomass 124 (fiber source) under conditions suitable to convert at least a portion of the biomass to sugars, thereby forming a mixture of solid, liquids, and dissolved sugars. Much of the dissolved sugars are results of biomass conversion and/or saccharification and are cellulosic sugars and sugar oligomers.

In some embodiments, the treatment conditions include wet milling 112 of the cellulosic biomass prior to or in combination with the treatment step 113. In some embodiments, the wet milling 112 is performed in a high shear reactor as described herein to enhance the saccharification rates. The biomass mixture can be diluted with backset 147 or centrate liquids if desired to reduce the solids content and the effective viscosity of the mixture. The biomass mixture can have, for example, a solids content of at least about 5%, 10%, 15%, 20%, 25%, 30%, 40% or 50% weight or greater depending on the effectiveness of the separation step and the amount of fiber source or cellulosic biomass added and its feed moisture concentration. The treatment conditions can include contacting the biomass mixture with cellulase and xylase enzymes 121 to hydrolyze components of the cellulosic biomass to sugars. The wet milling 112 and hydrolysis treatment 113 steps can be performed separately or combined. In some embodiments, the steps are combined in a shear reactor, such as an auger, that can manage continuous treatment with very high solids levels. The conditions can also include optimization of temperate and pH for saccharification and to minimize downstream impacts on the value and quality of co-products such as crude corn oil, animal feed, purified organic acids, and glycerol. The treatment step 113 can be any combination of equipment and technologies to effectively convert the cellulosic biomass into cellulosic sugars and sugar oligomers. The physical, chemical, thermal, and mechanical treatment conditions are tailored to enhance cellulosic sugars production and minimize inhibitor production.

The biomass mixture 149 after hydrolysis, conversion or saccharification is then contacted with the non-cellulosic feedstock 101 comprising starch and/or non-cellulosic sugars to achieve the high sugars concentration desired for fermentation. In some embodiments, the hydrolyzed biomass mixture is separated by a separations step B 114 into a liquid stream B 150 and a solids stream B 151 using separation methods known in the art and described herein. For example, the biomass mixture 149 can be separated with flotation, presses, screens, filters, or membranes or any combination. In some embodiments, the liquid stream B 150 is contacted with the non-cellulosic feedstock slurry. The liquid stream B 150 comprises sugars that are added to the upstream feedstock slurry, and the slurry stream 141 is treated to produce a mash 142 as described above, thereby completing the cycle. Thus, for a given solids concentration in the liquefied mash 142 feedstock, the addition of sugars derived from the cellulosic biomass 124 allows for increased ethanol production during fermentation 106 and or the reduction in feedstock 101 for constant ethanol production during fermentation. In other words, the method allows for the use of less corn or other non-cellulosic feedstock 101 to obtain the same sugars concentration in the fermentation or conversion step 106, because some of the sugars are now derived from cellulosic biomass, and not from starch. In some embodiments, the liquid stream B 150 comprises 1 to 8% w/v sugars. The backset 147 can be used as a source for the moisture or water needed for the treatment 113 or cellulosic biomass conversion process, prior to the non-cellulosic feedstock slurry 104 and liquefaction 105 processes. The ratio of cellulosic sugars to non-cellulosic sugars is a result of the ratio of cellulosic biomass 124 to non-cellulosic feedstock 101 and the conversion efficiencies of the treatment 113 and separation 114 steps and the slurry 104 and liquefaction 105 steps. For example, in some embodiments the ratio of cellulosic biomass to non-cellulosic feedstock is about 0.01, 0.03, 0.04, 0.06, 0.08, 0.10, 0.12, 0.14, 0.20 or greater. In some embodiments the separations step B 114 can include multiple or secondary processes to enhance operations. For example the liquid stream B 150 can include secondary membrane separations (filtration, micro-filtration, nano-filtration, and reverse osmosis) comprising process to concentrate the sugars, to isolate inhibitors, to recycle active components such as enzymes and other saccharification enhancing components, and to eliminate fine suspended particles.

In one embodiment, the liquid stream A 146 or a portion thereof is passed to an evaporator 116 where thin stillage 148 is evaporated to recover water 152 and to produce a concentrated stillage and eventually syrup 153. The portion of the liquid stream A 148 passed to evaporation is sometimes defined as thin stillage. The evaporated thin stillage can be processed to produce one or more co-products (such as Products B 128 and Product C 129) including oil, syrup, and/or DDGS. For example, the evaporated thin stillage can be separated 117 into a corn oil product (Product C 129) and an evaporated thin stillage stream or syrup 153 that is combined with the wet grains 145 and dried 118 to produce DDGS (Product B 128). The water 152 recovered from the evaporators (i.e., “cook water” 115) can be used in any or all of steps comprising to dilute 155 the hydrolyzed biomass or to dilute the biomass solids during the wet milling and/or treatment step or to wash the solids stream B or C and or added to the feedstock slurry 160. Make up water 130 can be added to the cook water as desired. Emissions from both the driers 118 and the distillation 108 and purification step 109 can be monitored and controlled.

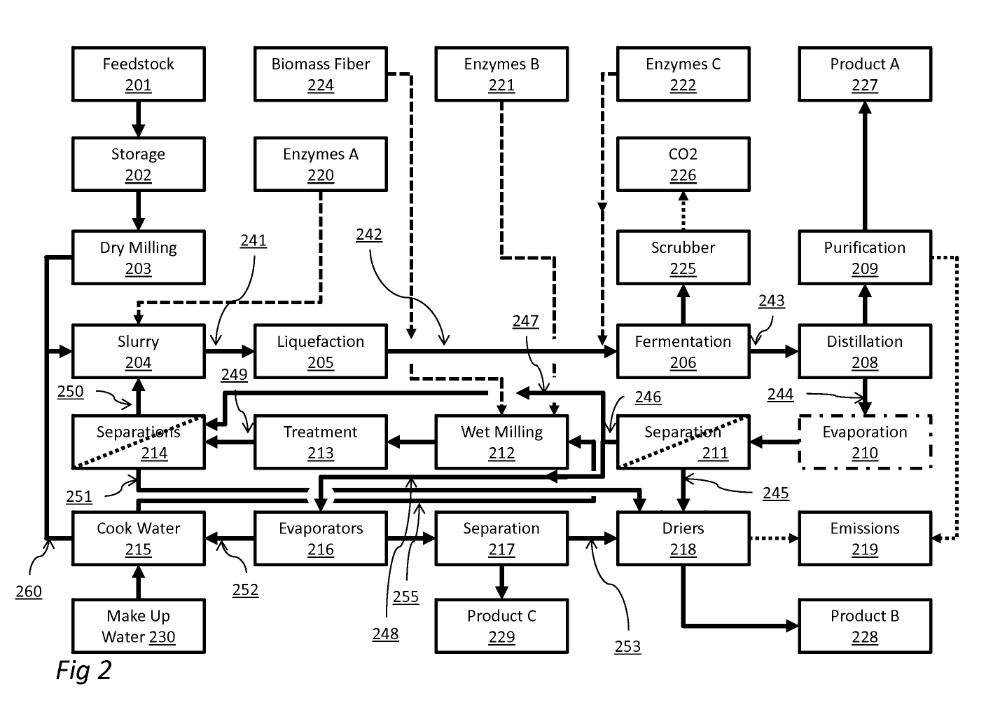

Referring now to FIG. 2, another illustrative embodiment will be described. The steps illustrated in FIG. 2 are similar to the embodiment shown in FIG. 1 where item 2XX is similar to item 1XX in FIG. 1. In the embodiment illustrated in FIG. 2, the liquid stream A 246 from the whole stillage 244 separation step A 211 is divided into two steams, a first portion and a second portion. One stream 247, referred to as backset or the first portion of the liquid stream A, is used to dilute the hydrolyzed biomass mixture 249 prior to the separation step B 214 or to wash the solids stream B 251 after separation or to add to slurry. The mixture 249 is separated into a liquid stream B 250 and a solid stream B 251 as described above, and the liquid stream B 250 comprising the cellulosic sugars is added to the feedstock slurry 204. As described above, the additional sugars derived from the cellulosic biomass increase the sugars concentration of the slurry stream 241, the liquefaction step 205, and post liquefied stream 242, and thereby, the ethanol yield during fermentation 206 and/or maintain constant sugars concentration and allow less corn kernel feedstock 201 to be used to produce a similar amount of ethanol 227. Enzymes 222 can be added to fermentation along with yeast and the post fermentation mash 243 can be distilled 208 and purified 209 to recover a product 227 such as ethanol. The other stream 248 (i.e., thin stillage) or the second portion of the liquid stream A, is evaporated 216 to recovery water 252 and to produce an evaporated thin stillage. The evaporated thin stillage is processed 217 to produce one or more products including oil 229 and de-oiled syrup 253 which can be mixed with wet grains 245 and dried 218 to DDGS 228, as described above. The water recovered from the evaporators 252 (i.e., “cook water” 215) or at least a portion of the water recovered 255 is recycled to dilute the biomass solids during the wet milling 212 and/or treatment step 213 either which include the addition of saccharification enzymes 221. Make up water 230 can be added to the cook water as desired. In addition, a portion of the water recovered can be used to wash (not illustrated) the fiber source or cellulosic biomass or to wash the second solids stream 251 after or during the second separation step 214. The post washing streams can be treated to recovery sugars or to extract inhibitors prior to being combined with the wet milling 212 and/or treatment 213 step or slurry 204 step. The recovered water 252 or a portion of the recovered water can be used as the source for the moisture or water needed for the treatment 213 or biomass conversion process and/or washing steps after separations but prior to the slurry 204 and liquefaction 205 process. In these embodiments the co-processing of cellulosic biomass 224 and non-cellulosic feedstock 201 comprising non-cellulosic material is enhanced by processing the cellulosic biomass 224 in the recycled and recovered water sources form the conventional feedstock processing steps before the cellulosic sugar enriched streams 250 are used for the slurry 204 and liquefaction 205 steps of conventional process, and thereby, eliminating extra fermentation or conversion vessels or reactors and downstream product recovery equipment because of the co-fermentation and/or co-conversion of both cellulosic and non-cellulosic sugars in the original equipment or co-processing equipment. In some embodiments either the backset 247 or recovered water streams 252 or a combination of both can be used for the various dilutions, washing, filtering, conversion, and/or other procedures. The feedstock 201 can be stored 202 and dry milled 203 to flour and mixed with cook water 260 in the slurry step 204 in which enzymes 220 are added. Gaseous products of fermentation step 206 can be scrub 225 prior to recovery or release of the co-product 226. Emissions 219 from the driers 218 can be managed, and the whole stillage stream 244 can be processed by an optional evaporation step 210 prior to the decanter separation step 211.

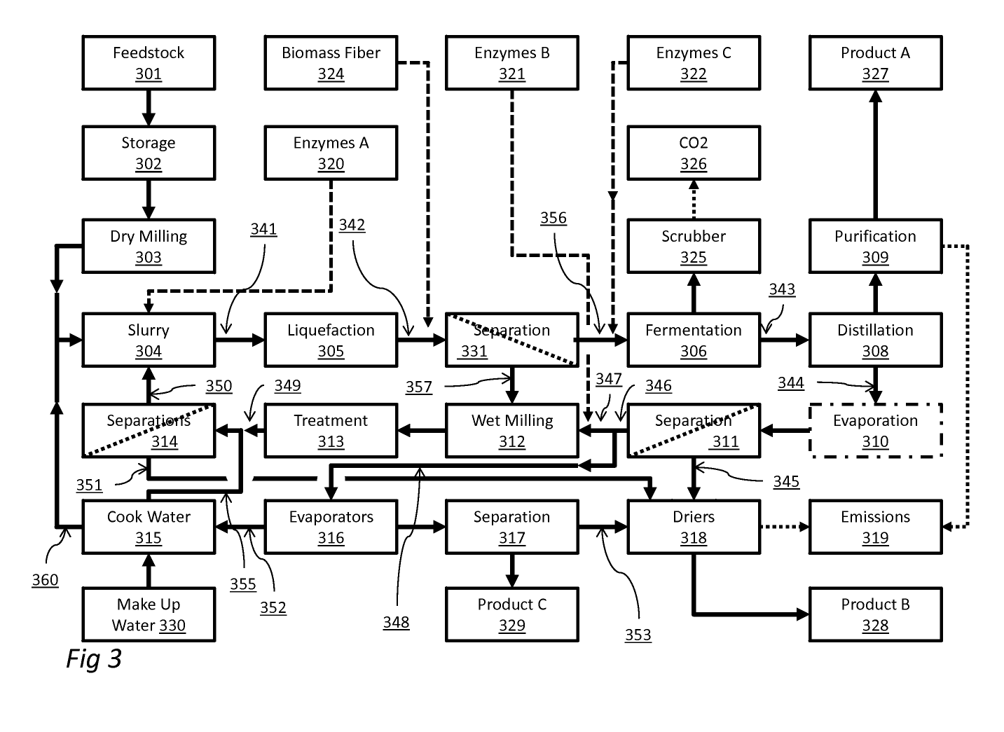

Referring now to FIG. 3, another illustrative embodiment will be described. In this embodiment, the liquefied mash 342 is filtered through a filter 331 comprising cellulosic biomass solids 324, such as cellulosic biomass and/or biomass fibers. In some embodiments, the biomass solids 324 comprise dry fiber or corn stover. The biomass solids can be incorporated into a filter device, such as but not limited to a filter press, extruding press, vibrating filter press, Vincent type press, belt filter press, vacuum filter press, cylinder press, sand-type filter, or any other suitable filter known in the art or combination of these devices. The biomass fibers 324 can be pretreated or non-pretreated biomass. The biomass filter 331 separates the liquefied mash 342 into a liquids C stream 356 comprising dissolved sugars and a solids stream C 357 comprising non-soluble corn solids and cellulosic biomass. In some embodiments, the liquid stream C 356 is a relatively clear liquid stream comprising high concentrations of dissolved sugars that is sent to the fermenters 306. In some embodiments, the concentration of sugars or fermentable sugars in the liquid stream C is at least 10%, 20%, 30%, 40%, and 50%, sugars or sugar oligomers based on the solids level of the liquefied mash 342. The liquids stream C 356 will also have relatively low amounts of non-fermentable solids, which allows for increased amounts of ethanol 327 to be produced during fermentation 306, shorter fermentation cycle times, higher yields and/or lower non-fermentable solids in the mash. Without being bound by theory, it is believe that decreasing the dissolved non-fermentable solids in the fermentation step 306 prevents osmotic stress on the yeast that occurs when the solids concentration is above about 30-35% w/v. In some embodiments, the amount of ethanol 327 produced by the method is at least 1%, 3%, 5%, 10%, 15%, 20%, or more than the amount of ethanol produced by a method that does not include the step of filtering the mash through a biomass fiber filter 331. Improvements in ethanol product 327 can result from higher conversion efficiencies, shorter cycle times, and increased sugars concentrations. The post fermentation mash 343 is typically distilled 308 and purified 309 to generate a product 327 and a whole stillage stream 344 with relatively low solids which can be optionally evaporated 310 before separation step 311. Any co-feed cellulosic material 324 can be used in a process in which the post liquefied mash stream 342 is separated into the liquid stream C 356 and the solids stream C 357. Surprising advantages occur with both streams from this separation step C 331. The liquid stream C 356 is very low in suspended solids which provides various optimization in fermentation and downstream of fermentation. Because of the low suspended solids, extraction of the yeast cells and yeast cell extracts are feasible, as well as recovery of byproduct of fermentation such as glycerol, phosphates, nitrates, and organic acids. The solids stream C 357 comprises most of the non-starch components of the feedstock, such as corn flour, and all or most of the co-feed cellulosic biomass. Processing of this solids stream C 357 can be achieved independent of the fermentation process 306 with various steps to generate cellulosic sugars and recovery high value co-products such as animal feed and oil. The fermentation step 306 can be enhanced with other enzymes 322 and the gaseous co-product can be scrub 325 before recovery or releasing carbon dioxide 326.

The solids stream C 357 typically comprises non-dissolved components from the liquefied mash 342, such as non-soluble proteins, fats, and non-hydrolyzed starch from the feedstock grain 301. In some embodiments, the solids stream C 357 also comprises the biomass solids/fibers 324, for example corn stover, used in the filter. In some embodiments, the solids stream C 357 is mixed with the centrate stream 346 from the decanter centrifuge 311 or a portion 347 of the centrate stream to produce a second mash or biomass mixture. In some embodiments, the solids stream C 357 is mixed with the backset 347, recovered water 352 and/or any combination or portion of these streams. The mixture is treated 313 with cellulase enzymes 321 and subjected to wet milling 312 and/or high shear reactors as described above to covert the cellulose fibers into shorter chain soluble sugars. The enzymes 321 can be added before or after the wet milling step 312. The mixture can also be treated or saccharified by other techniques to convert the cellulosic biomass into shorter chain soluble sugars, such as but not limited to acid treatments, basic treatments, physical and thermal treatments, etc. In some embodiments the hydrolyzed mixture 349 is separated 314 as described above into a liquid stream B 350 and a solids stream B 351. The liquid stream B 350 comprising the cellulosic sugars is combined with the feedstock 301 after dry milling 303 to form a slurry 304 and slurry stream 341, and the process is repeated. Thus, the process can be a continuous process and the first liquefied stream used to generate the solids stream C is the same as the second liquefied stream generated from mixing the liquid stream B with feedstock. In other embodiments, the process is a batch process and or the feedstock 301 can be stored 302. In some embodiments the liquid stream B 350 can also include smaller amounts of non-cellulosic sugars recovered from the liquid content of the solids stream C 357 or from various wash steps or hydrolysis of any residual starch captured in the solids stream C 357 in the filter or corn solids.

In some embodiments the corn mash solids or residual non-cellulosic feedstock solids after liquefaction can be further separated from the cellulosic biomass 324 generating a first portion of the solids stream C comprising cellulosic biomass and a second portion of the solids stream C, comprising mash solids. In these embodiments, the first portion of the solids stream C can be treated 313 separately or together form the second portion of the solids stream C. For example, a filter press using a dual stroke cylinder mechanism can achieve this secondary separation by first pressing a fiber mat with a first stroke followed by the addition of the liquefied mash 342 between the fiber mat and the piston and then followed by a second stroke to press the liquefied mash 342 through the fiber mat and removal of the liquid stream C 356 thorough an outlet. When the second stroke is complete, a gate is opened and the fiber mat is pushed out with a first stroke extension and removed and then followed by a final extension of the piston, which is used to push the second portion of the solids stream C out for removal. The piston retracts and the dual cycle is repeated.

In this embodiment, the types and number of steps for the treatments 313 and 312 and the conditions of treatment can vary between the first and second portion of the solids stream C. For example, the second portion of the solids stream C can be washed to recover and recycle non-cellulosic sugars in the liquid contained in the solids or the stream can be treated with cellulase enzymes 321 designed to aggressively hydrolyze the germ cells and oil emulsion components to enhance downstream oil recovery (discussed later), while achieving a mild conversion of the corn kernel fiber content without over treating or damaging the protein material. In parallel, the first portion of the solids stream C can be passed to saccharification treatment 313 or can be aggressively pretreated and wet milled 312 to prepare the cellulosic biomass for saccharification. Higher levels of enzymes 321 can be used to enhance and maximize saccharification of the first portion or the first portion can be saccharified by an alternate process, such as but not limited to acid hydrolysis. The first portion and second portion can continue to be treated separately or can be combined at any step prior to introduction into the slurry stream 341. If an acid is used the recovered liquid from this process with residual acid can be used in other process steps to adjust the pH of the process streams.

As described above, in some embodiments, the centrate stream 346 or a portion thereof, comprising thin stillage 348, is evaporated 316 to produce an evaporated thin stillage or syrup 353 which can be mixed with the solids stream B 351 and or the solids stream A 345 and dried 318 to produce a product 328 such as DDGS. The evaporated or partially evaporated thin stillage stream can be further processed by separation step 317 to recovery co-products 329 such as corn oil, glycerin, acetic acid, and others. The water recovered 352 from the evaporators can be used to dilute the cellulosic biomass mixture, to wash various solids steams, or added to the feedstock slurry. The recovered water 352 or cook water 315 can be mixed with make-up water 330 and or passed to slurry 304 as dilution water 360. In some embodiments, the solids stream B 351 is dried 318 to produce a product (Product B 328). These water streams or some mixture of these streams 355 can also be used to enhance separation step 314 and the wet milling steps 312 and treatment step 313. Amylase enzymes 320 are typically added to slurry step 304 to enhance liquefaction step 305. Emissions 319 from the driers 318 and distillation and purification steps 309 can be managed.

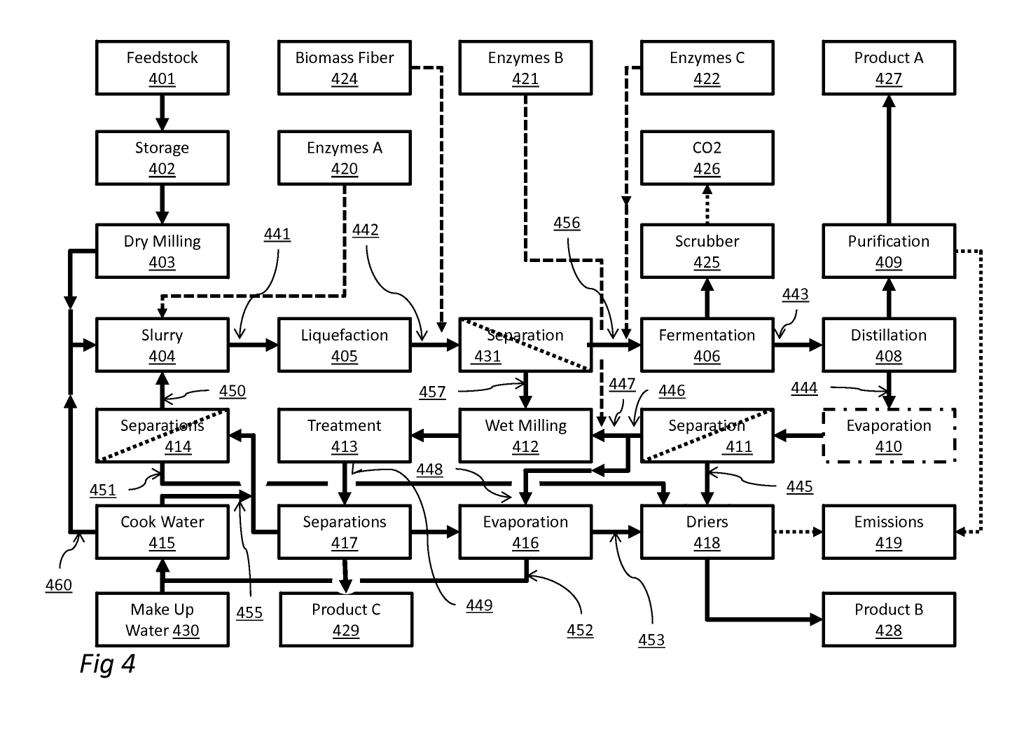

Referring now to FIG. 4, another illustrative embodiment will be described. The embodiment illustrated in FIG. 4 is similar to the embodiment shown in FIG. 3, with the additional separation step D 417 of recovering a co-product 429 (Product C) from the hydrolyzed biomass mixture 449. In one embodiment, the product 429 is oil, for example, corn oil and because this recovery step 417 is occurring in a stream with all of the oil in the feedstock 401 the overall recovery rates will be substantially greater. The product can be recovered in one or more separation steps 417. For example, the post-treatment mixture 449 (e.g., the post-saccharification mixture or post conversion mixture depending on the type of treatment) can be centrifuged to recover relatively pure crude oil (i.e., a stream comprising at least 50%, 60%, 70%, 80%, 90%, 95%, 98%, 99% or 100% oil), or an oil/emulsion stream that can be further processed into a marketable oil product 429. In one embodiment, a tri-phase style centrifuge (e.g., a decanter or disk centrifuge depending on feed consistency) is used to generate a first stage recovery stream (light phase mixture), a soluble and fines suspended solids stream (medium phase mixture), and a high fiber solids stream (heavy phase mixture). These centrifugation steps can be integrated with other separation techniques. For example, the post treated mixture 449 can be dosed with a polymer compound or additive which when aerated with fine air bubbles forms a suspended solids float above the bulk liquid phase that can be recovery with a skimmer, often referred to as a dissolved air floatation (DAF) separation, and centrifuged to recover the co-product oil. Another example is the medium phase mixture from the centrifuge can be further processed using the above dissolved air flotation process to extract the suspended solids after oil recovery and processed as an animal feed co-product while the clarified liquid from the DAF can be subjected to membrane separation methods to isolate and/or concentrate the dissolved solids and sugars. The separation 414 which can be achieved with or without the DAF pre-separation produces a permeate stream comprising dissolved sugars 450 and a retentate stream comprising a solids (e.g., wet grains) stream 451. In some embodiments, the membrane separation comprises a first step to separate the suspended particles from a clear liquid stream comprising dissolved compounds, followed by a RO membrane step to separate the dissolved organic acids, inhibitors, glycerol, alcohols, and other permeable compounds from the larger ring structured sugars. The sugar stream can be combined or concentrated (e.g., by vapor compression or other low temperature distillation techniques) and combined with the feedstock slurry 404 produced by a conventional corn ethanol facility. The heavy phase mixture form the tri-phase centrifuge can be recycled back into the solids stream C 457 or the first portion of the solids stream C to effectively recover and recycle the cellulase enzymes 421 used in the treatment 413, conversion, or saccharification step designed to convert cellulosic biomass into cellulosic sugars and oligomers. The method provides surprising results in the number, magnitude, and efficacy of the enhancements which are achievable with the integration of various separations techniques and process treatment steps to maximize the number of co-products and the efficiencies of co-product production and/or recovery without impacting the optimizations and enhancements also achievable in the fermentation 406 or sugars conversion processes and product portfolios that can be achieved with the liquid stream C 456.

The overall process of FIG. 4 is similar to the processes described in FIGS. 1, 2 and 3 the feedstock 401 can be stored 402 prior to dry milling 403 into a flours. This feedstock 401 mixed with dilution water 460, cellulosic sugar rich liquids stream B 450 and enzymes 420 in the slurry step 404 to generate a slurry stream 441 that is passed to liquefaction step 405 to convert the feedstock starch to non-cellulosic sugars and sugar oligomers generating a liquefied stream 442. The separation step C 431 using biomass fibers 424 generates the solids stream C 457 comprising non-sugar components of the feedstock 401 and fibers 424 and a liquid stream C 456 which is mixed with yeast and additional enzymes 422 for the fermentation step 406. The post fermentation mash 443 which is low in suspended solids because of the separation step C 431 is passed to distillation 408 for recovery of the primary product 427 such as ethanol after purification step 409. Gaseous co-products of fermentation step 406 are scrubbed 425 before the carbon dioxide 426 is recovered or release. The yeast in the post fermentation mash 443 can be recovered and recycled before distillation. The post distillation mash or whole stillage 444 can be evaporated with optional evaporator 410 before being passed to the separation step A 411. The liquid stream A 446 can be separated into a backset streams 447 and or thin stillage stream 448. Either or both of these streams can be used for various wash and dilutions steps around the wet milling step 412, treatment step 413, and separations step B 414 or separation step C 431. The thin stillage stream can be mixed with some of the medium and heavy solids stream from the separation step D 417 and evaporated 416 to generate a syrup stream 453. This syrup stream 453 can be mixed with the solids stream B 451 from separations step B 414 and dried 418 to generate a co-product 428 such as DDGS. The solids stream A 445 can be recovered as yeast extracts or can be mixed with the solids stream B 451 and syrup 453. Emissions for the drier step 418 and purification step 409 can be managed. Water 452 recovered from evaporation can be mixed with make-up water 430 if needed to generate cook water 415 which can be passed as dilution water 460 or wash water 455 in separations step B 414, separation step C 431 or wet milling 412 and treatment 413 steps. Various washing and dilutions embodiments around the various liquid stream (146, 147, 148, 152, & 155; 246, 247, 248, 252, & 255; 346, 347, 348, 352, & 355; and 446, 447, 448, 452, & 455) discussed in FIGS. 1, 2, 3 and 4 are applicable for other embodiments defined in the other figures.

It will be understood that in the embodiments described above, the terms “first,” “second,” and “third,” when referring to solid and liquid streams, are for illustrative purposes only, and are not intended to limit or be construed as equivalent to the same term(s) in the claims.

### C. Separation Methods

The methods described herein make use of various types of separators and separation methods for example but not limited to 111, 211, 311, 411, 114, 214, 314, 414, 117, 217, 317, 417, 331, and 431. In some embodiments, the separator is a mechanical device, including but not limited to dissolved air flotation, decanting volume, crystallization, centrifuge, a decanter centrifuge, a disk stack centrifuge, or a press or combination of these techniques and processes. In some embodiments, the separator is a filter, such as but not limiting examples include a fiber bed, filter press, belt type press, Vincent type press, cylinder press, extruders, or sand-type filter. In some embodiments, the separator is a screen type separator. Non-limiting examples of screen type separators include screens, vibrating screens, reciprocating screens (rake screens), gyratory screens/sifters, and pressure screens. In some embodiments the separation process is aided with the addition of a biomass fiber to function as a binding agent or media to enhance or improve the cost effectiveness of the separation steps.

In some embodiments, the separator is a membrane type separator designed to manage various levels of suspended solids such as the SmartFlow membrane (Edeniq, Visalia, Calif.). Examples of membrane type separators include ultrafiltration (UF) membranes, microfiltration (MF) membranes, and Tangential Flow Filtration (TFF) systems and specific membranes can have different surface and bulk characteristics including hydrophobic and hydrophilic surfaces and/or tortuous flow paths and can be composite membranes with multiple layers to enhance performance.

MF membranes typically have a pore size of between 0.1 micron and 10 microns. Examples of microfiltration membranes include glass microfiber membranes such as Whatman GF/A membranes. UF membranes have smaller pore sizes than MF membranes, typically in the range of 0.001 to 0.1 micron. UF membranes are typically classified by molecular weight cutoff (MWCO). Examples of ultrafiltration membranes include polyethersulfone (PES) membranes having a low molecular weight cutoff, for example about 10 kDa. UF membranes are commercially available, for example from Synder Filtration (Vacaville, Calif.).

Filtration using either MF or UF membranes can be employed in direct flow filtration (DFF) or Tangential Flow Filtration (TFF). DFF, also known as dead end filtration, applies the feed stream perpendicular to the membrane face such that most or all of the fluid passes through the membrane. TFF, also referred to as cross-flow filtration, applies the feed stream parallel to the membrane face such that one portion passes through the membrane as a filtrate or permeate whereas the remaining portion (the retentate) is recirculated back across the membrane or diverted for other uses. TFF filters include microfiltration, ultrafiltration, nanofiltration and reverse osmosis filter systems. The cross-flow filter may comprise multiple filter sheets (filtration membranes) in a stacked arrangement, e.g., wherein filter sheets alternate with permeate and retentate sheets. The liquid to be filtered flows across the filter sheets, and solids or high-molecular-weight species of diameter larger than the filter sheet's pore size(s), are retained and enter the retentate flow, whereas the liquid along with any permeate species diffuse through the filter sheet and enter the permeate flow. The TFF filter sheets, including the retentate and permeate sheets, may be formed of any suitable materials of construction, including, for example, polymers, such as polypropylene, polyethylene, polysulfone, polyethersulfone, polyetherimide, polyimide, polyvinylchloride, polyester, etc.; nylon, silicone, urethane, regenerated cellulose, polycarbonate, cellulose acetate, cellulose triacetate, cellulose nitrate, mixed esters of cellulose, etc.; ceramics, e.g., oxides of silicon, zirconium, and/or aluminum; metals such as stainless steel; polymeric fluorocarbons such as polytetrafluoroethylene; and compatible alloys, mixtures and composites of such materials. Cross-flow filter modules and cross-flow filter cassettes useful for such filtration are commercially available from SmartFlow Technologies, Inc. (Apex, N.C.). Suitable cross-flow filter modules and cassettes of such types are variously described in the following United States patents: U.S. Pat. No. 4,867,876; U.S. Pat. No. 4,882,050; U.S. Pat. No. 5,034,124; U.S. Pat. No. 5,034,124; U.S. Pat. No. 5,049,268; U.S. Pat. No. 5,232,589; U.S. Pat. No. 5,342,517; U.S. Pat. No. 5,593,580; and U.S. Pat. No. 5,868,930; the disclosures of all of which are hereby incorporated herein by reference in their respective entireties.

In some embodiments, the separator is a reverse osmosis (RO) type separator. Examples of RO type separators include RO spiral membranes available from Koch Membrane Systems (Wilmington, Mass.) or Synder Filtration (Vacaville, Calif.). RO type separators are more effective at separations at the small molecular scale, such as extracting inhibitors form sugars and sugar oligomers.

### D. Saccharification and Fermentation Conditions

The saccharification reaction can be performed at or near the temperature and pH optimum for the saccharification enzymes used. In some embodiments of the present methods, the temperature optimum for saccharification ranges from about 15 to about 100° C. In other embodiments, the temperature range is about 20 to 80° C., about 35 to 65° C., about 40 to 60° C., about 45 to 55° C., or about 45 to 50° C. The pH optimum for the saccharification enzymes can range from about 4.0 to 7.0, about 4.0 to 6.5, about 4.0 to 6.0, about 4.5 to 6.0, about 4.0 to 5.5, about 4.5 to 5.5, or about 5.0 to 5.5, depending on the enzyme.

Examples of enzymes that are useful in saccharification of lignocellulosic biomass include glycosidases, cellulases, hemicellulases, starch-hydrolyzing glycosidases, xylanases, ligninases, and feruloyl esterases, and combinations thereof. Glycosidases hydrolyze the ether linkages of di-, oligo-, and polysaccharides. Enzymes can also include inulases for inulin containing feedstock. The term cellulase is a generic term for a group of glycosidase enzymes which hydrolyze cellulose to glucose, cellobiose, and other cello-oligosaccharides. Cellulase can include a mixture comprising exo-cellobiohydrolases (CBH), endoglucanases (EG) and β-glucosidases (βG). Specific examples of saccharification enzymes include carboxymethyl cellulase, xylanase, β-glucosidase, β-xylosidase, and α-L-arabinofuranosidase, and amylases. Saccharification enzymes are commercially available, for example, Pathway™ (Edeniq, Visalia, Calif.), Cellic® CTec2 and HTec2 (Novozymes, Denmark), Spezyme® CP cellulase, Multifect® xylanase, and Accellerace® and Accellerace Trio® (DuPont Industrial Biosciences, Rochester, N.Y.). Saccharification enzymes can also be expressed by host organisms, including recombinant microorganisms.

The enzyme saccharification reaction can be performed for a period of time from about several minutes to about 250 hours, or any amount of time between. For example, the saccharification reaction time can be about 5 minutes, 10 minutes, 30 minutes, 60 minutes, or 2, 4, 6, 8, 12, 16, 18, 24, 36, 48, 60, 72, 84, 96, 108, 120, 130, 140, 150, 160, 170, 180, 190, 200, 210, 220, 230, 240 or 250 hours. In other embodiments, the saccharification reaction is performed with agitation to improve access of the enzymes to the cellulose.

The amount of saccharification enzymes added to the reaction can be adjusted based on the cellulose content of the biomass fibers and/or the amount of solids present in a composition comprising the biomass or biomass mixture, and also on the desired rate of cellulose conversion. For example, in some embodiments, the amount of enzymes added is based on percent by weight of cellulose present in the biomass, as specified by the enzyme provider(s). The percent of enzyme added by weight of cellulose in such embodiments can range, for example, from about 0.1% to about 10% on this basis.

In some embodiments, the hydrolysis is performed in a reaction vessel. In some embodiments, the reaction vessel is a mixing device or a high shear mixing device as described herein. In one embodiment, the reaction vessel is an auger. In some embodiments, the hydrolysis reaction occurs under conditions of counter-current flow, such that the solids are transported in a different or opposite direction than the liquids. Counter-current flow has the advantage of separating liquids containing sugars from the non-hydrolyzed solids, thereby lowering the local concentration of sugars that can inhibit hydrolytic enzymes. In one embodiment, the counter-current flow occurs in an auger.

After the biomass is pretreated and hydrolyzed as described herein, the sugars can be used for any desired downstream process or refined as a product. In one embodiment, the sugars are fermented to ethanol, as described below.

After the saccharification steps described above, the treated biomass mixture and/or converted sugars can be subjected to fermentation under conditions sufficient to produce ethanol from the sugars. The fermentation conditions include contacting the biomass and/or sugars with yeast that are capable of producing ethanol from sugars. If desired, the biomass can be subjected to simultaneous saccharification and fermentation (SSF). The pH of the SSF reaction can be maintained at the optimal ranges for the activity of the cellulosic enzymes, for example between 4.0 to 7.0, about 4.0 to 6.5, about 4.0 to 6.0, about 4.5 to 6.0, about 4.0 to 5.5, about 4.5 to 5.5, or about 5.0 to 5.5.

Fermentation can also produce alcohol, organic acids, acetone, butanol or other products that are removed from the fermentation broth by a process comprising distillation or evaporation of the product. The remaining material after distillation or evaporation, the “stillage,” can then be treated to recover residual oil and/or other desired products from the stillage. Other desired products can include, but are not limited to, organic acids, lipids or oils, glycerin, sugars, proteins, amino acids, soluble/insoluble fiber including polysaccharides and oligosaccharides, and others. Examples of organic acids that can be recovered, depending on the particular fermentation and the microorganisms involved, include acetic, lactic, succinic, oxalic, citric, malic, formic, propionic, butyric acid, and others.

The cellulosic and non-cellulosic sugars and oligomers can be converted to “ethanol” or other “product(s)” such as but not limited to methanol, butanol(s), propanol(s), aromatics, farnesene, acetic acid, lactic acid(s), levulinic acid(s), succinic acid(s), isoprene(s) and others during fermentation, or that can be converted to synthetic gases comprising hydrogen and carbon monoxide, which can be converted to fuels such as but not limited to naphtha, kerosene, gasoline, and diesel replacements, or chemical products, such as but not limited to waxes, acetic acid, formaldehydes, polyethylene, xylenes, alcohols, oxygenates, synthetic LPG, olefins, ammonia, fertilizers, industrial chemicals, fine chemicals, and petroleum replacements chemicals, and to electric power and other energy media.

### EXAMPLES

### Example 1

This example illustrates that adding sugars derived from cellulosic biomass can be used to reduce the total amount of solids in fermentation while maintaining the sugars concentration.

One type of optimization is to reduce the total solids in fermentation while maintaining the same sugars concentration in the post liquefied mash and thereby, increase process efficiency by reducing osmotic pressure on the yeast organisms. In a baseline corn ethanol plant the solids and sugars target can be approximately 33% slurry total solids, 31% slurry corn solids (extracting 2% solids introduced with backset stream) and 24% post liquefaction sugars solids after hydrolysis (31%*70% starch/corn*1.11 sugar/starch). If the co-feed cellulosic sugars represent 3% post liquefaction sugars solids, then the corn solids could be reduced to 27% slurry corn solids ((100%-3% cell sugars/24% target sugars)*31% slurry corn solids) or a 12% reduction in corn solids. With the separation step after saccharification of the co-feed cellulosic biomass that removes the non-dissolved solids prior to passing the cellulosic sugars to the slurry stream, the reduction in corn solids results in a reduction in total solids by about 1%, as illustrated by the calculation (31% corn solids*(100%-70%) non starch solids*12% reduction), while still maintaining the 24% post liquefied sugars solids.

### Example 2

This example shows that biomass fibers can be used to filter the post-liquefied mash and recover sugars.

To evaluate the effectiveness of filtering the post liquefied mash with fiber to generate a clarified sugars stream and a solids stream, a filter test was conducted. A sample of 105.7 gm of post liquefied mash with 30% w/w corn solids with a 65% dry w/w starch composition (74.0 gm water, 31.7 gm corn solids) was filtered through 58.5 gm of wet cellulosic biomass with 10% w/w solid (5.85 gm fibers and 52.7 gm moisture), for a total system mass of 164.2 gm. A plug press filter was used and resulting in a three phase product consisting of 37.9 gm wet of corn solids plug, 13.8 gm wet fiber solids plug, and 112.5 gm of liquid (91.5% directly recovered). The solids of each phase was measured resulting in corn solids plug at 39.5% w/w solids, fiber plug at 48.3% w/w solids, and recovered liquid at 14.1% w/w solids and on balance 100.1% of the water and 99.8% of the solids were recovered. Assessing the change in dry mass solids 52.8% of the corn solids were lost from the corn plug (change from 31.75 gm to 14.96 gm dry) and the fiber plug gained about 14% (change 5.85 gm to 6.67 gm dry). Assuming only starch mass was related to these changes (short and med chain sugar oligomers), 19% of the starch remained in the corn plug, 4% was in the fiber plug, and 77% was in the liquid phase. Examining the sugar composition of the post liquefied corn mash and the recovered liquid provided similar results. The HPLC analysis of the slurry indicated that 76% of the measured dissolved compounds were DP-4/Dextrin, 9% were DP-3/Maltoriose, 10% were DP-2/Maltose, and 4% were DP-1/Glucose, and about 0.4% Glycerol, while the recovered liquid indicated that 78% was DP-4, 9% DP-3, 9% DP-2, 4% DP-1, and 0.3% Glycerol. Analysis of this data indicated that 72% to 85% of the individual sugars were recovered and in total about 65% w/w was recovered. It is believed that a water wash and secondary pressing of the solids stream (corn plug and fiber plug) would have improved these recovery factors.

### Example 3

This example illustrates the economic advantages of the co-feed methods as integrated into a conventional corn ethanol facility, which produces 110 MGPY of denatured ethanol at a yield of 2.75 gal of non-cellulosic ethanol per bushel of corn and needs 40M bushels of corn per year. The ethanol is sold at $2.25/gal for $275.5 M per year revenue, while the corn costs $6/bu for a cost of $240M per year. The co-products include 316 k tons of DDGS which is sold at $182/ton for a $57.6 M per year revenue. Factoring the cost of natural gas and electricity, enzymes, and other cost of goods, the net EBITDA (earnings before interest, taxes, depreciation and amortization) is about $24.3M per year.

If the facility installs a conventional corn oil recovery system with a performance of 0.55 lb of oil/bushel of corn, the EBITDA increases to $31.1 M per year due to $8.8M/year in oil sales and the loss of $2.0M/year in DDGS sales and minor changes in energy and other related costs.

Installing a Cellunator™ system to achieve higher yields up to 2.83 gal/bu of corn further improves the plant's EBITA to $35.0M/year by decreasing the corn cost by $7.0M/year and increasing oil sales by $2.3M/yr and maintaining the same ethanol output, but losing another $5.0M in DDGS due to lost mass. Natural gas and enzyme savings basically offset other improvements.

Next if the plant implements the first stage Pathway™ product line to achieve an additional 2% increase in yield from corn kernel fiber conversion to 2.89 gal/bu, its EBITA increases to $39.5M/year, from a $4.6M/yr decrease in corn costs partially offset by a further decrease in DDGS due to lost mass of $3.9M/year. Additional oil production adds another $2.8M/yr and increased government credits of $2.16M/yr.

Finally by implementing the co-feed option using only some of these embodiments the plants EBITA can be increased to $50.7M/yr. Assuming 4% of the ethanol is produced from co-feed cellulosic biomass the efficiency is increased to 3.0 gal/bu of corn providing a $9.1M/yr corn cost saving while adding a $8.2M/yr cost for corn stover cellulosic biomass, and a $10.7M/yr increase in DDGS, recognizing increased mass from non-converted cellulosic biomass and reducing the protein content of the DDGS back to the level of the conventional facility. Increases in natural gas cost, operating costs, and cellulase enzyme cost help balance the $4.4M/yr increase in government credits for cellulosic ethanol. The $50.7M/yr represents a 108% increase in the baseline facility's EBITA and 28% increase for the small fraction of the embodiments implemented. If the enhanced oil recovery benefits are recognized by implementing other embodiments described herein, an additional $15 to 20M/yr EBITA improvement appears achievable which can relate to a 65% increase from these embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded.

What is claimed is:

1. A method for processing a cellulosic biomass, comprising:
a) generating a liquefied mash from a feedstock comprising non-cellulosic biomass;
b) filtering the liquefied mash through cellulosic biomass to generate a first liquids stream comprising dissolved sugars and a first solids stream comprising the cellulosic biomass and non-dissolved components from the liquefied mash;
c) treating the first solids stream under conditions sufficient to convert components of the biomass to cellulosic sugars, thereby producing a mixture comprising solids, liquids, and dissolved cellulosic sugars;
d) separating the mixture into a second liquids stream comprising dissolved sugars and a second solids stream;
e) contacting the second liquids stream with feedstock to form a slurry; and
f) processing the slurry to produce liquefied mash, thereby producing a mash comprising both cellulosic and non-cellulosic sugars.

2. The method of claim 1, further comprising processing the first liquid stream under conditions suitable to produce a product from the sugars and a whole stillage stream.

3. The method of claim 2, further comprising processing the whole stillage stream to generate a third liquids stream and a third solids stream, wherein a portion of the third liquids stream and the first solids stream are mixed under conditions suitable to convert components of the biomass to sugars.

4. The method of claim 3, wherein water is recovered from at least a portion of the third liquids stream and the water is mixed with the first solids stream under conditions suitable to convert components of the biomass to sugars.

5. The method of claim 1, wherein the mixture is treated with a high shear reactor.

6. The method of claim 2, wherein the first liquids stream is fermented to produce the product.

7. The method of claim 6, wherein the product is ethanol, succinic acid, butanol(s), methanol, propanol(s), isoprene(s), aromatics, farnesene, acetic acid, lactic acid(s), or levulinic acid(s).

8. The method of claim 1, further comprising recovering an oil co-product from the mixture.

9. The method of claim 1, wherein the filtering step comprises filtering the mash through biomass comprising fiber.

10. The method of claim 1, further comprising separating the mixture into a filtrate comprising sugars and a retentate comprising solids and enzymes and contacting a portion of the solids to step (c).

11. The method of claim 3, further comprising contacting at least a portion of the third liquids stream with the first solids stream prior to or during step (c) and/or step (d).

12. A method for producing ethanol from a cellulosic biomass in an ethanol facility, comprising:
a) separating a whole stillage into a first liquid stream and a first solids stream;
b) contacting the cellulosic biomass with at least a portion of the first liquid stream under conditions suitable to convert components of the biomass to sugars, thereby producing a mixture comprising solids, liquids and dissolved cellulosic sugars;
c) contacting the mixture with feedstock comprising non-cellulosic biomass to form a slurry; and
d) processing the slurry under conditions sufficient to produce ethanol, thereby co-producing ethanol from the cellulosic sugars and the non-cellulosic feedstock.

13. A method of claim 12, further comprising:
e) recovering water from at least a portion of the first liquid stream; and
f) contacting the cellulosic biomass with the recovered water under conditions suitable to convert components of the biomass to sugars, thereby producing a mixture comprising solids, liquids and dissolved cellulosic sugars.

14. The method of claim 12, further comprising:
separating the mixture into a second liquid stream comprising fermentable sugars and a second solids stream comprising non-converted biomass, and
contacting the second liquid stream with feedstock to form the slurry.

15. The method of claim 14, further comprising washing the second solids stream with an aqueous solution and adding the post-wash aqueous solution to the slurry.

16. The method of claim 14, wherein a portion of the second solids stream is contacted with the biomass under conditions suitable to convert components of the biomass to sugars, thereby producing sugars.

17. The method of claim 12, wherein the cellulosic biomass comprises corn stover, wheat straw, bagasse, wood or any other cellulosic fiber, and the cellulosic biomass is pretreated or non-pretreated.

18. The method of claim 12, wherein the feedstock comprises corn, wheat, milo, rice, barley, sugar cane, sugar beets, tubers or Jerusalem artichokes.

19. The method of claim 12, wherein the biomass and/or mixture is treated with a high shear reactor.

20. The method of claim 12, wherein the conditions suitable to convert include contacting the biomass with enzymes comprising cellulases such that the enzymes hydrolyze at least a portion of the biomass to sugars.
